(19) United States
(12) Patent Application Publication
(10) Pub. No.: US 2016/0024520 A1
(43) Pub. Date: Jan. 28, 2016

(54) CLONING AND USE OF THE MS9 GENE FROM MAIZE

(71) Applicants: PIONEER HI-BRED INTERNATIONAL, INC., Johnston, IA (US); E.I. DUPONT DE NEMOURS & COMPANY, Wilmington, DE (US)

(72) Inventors: Marc C. Albertsen, GRIMES, IA (US); TIM FOX, DES MOINES, IA (US); APRIL LEONARD, WILMINGTON, DE (US); BAILIN LI, HOCKESSIN, DE (US); BRIAN LOVELAND, COLLINS, IA (US); MARY TRIMNELL, WEST DES MOINES, IA (US)

(21) Appl. No.: 14/773,465

(22) PCT Filed: Mar. 14, 2014
(86) PCT No.: PCT/US14/27350

§ 371 Date: Sep. 8, 2015

Related U.S. Application Data
(60) Provisional application No. 61/801,289, filed on Mar. 15, 2013.

(51) Int. Cl. C12N 15/82

(52) U.S. Cl. CPC C12N 15/8289

(57) ABSTRACT

Nucleotide sequences, amino acid sequences, and an associated promoter sequence are useful in methods of controlling male fertility in plants. Recombinant expression cassettes, vectors, plant cells, and plants comprise a disclosed nucleotide sequence and may encode a disclosed amino acid sequence. The recombinant expression cassettes are useful in controlling fertility, especially male fertility of annual crops. The promoter is useful in driving expression of an operably-linked heterologous polynucleotide. Constructs comprising homology to the promoter sequence are useful for downregulation of a polynucleotide associated with the promoter.

### FIELD OF THE INVENTION

The present disclosure relates to the field of plant molecular biology, more particularly to influencing male fertility.

### BACKGROUND

Development of hybrid plant breeding has made possible considerable advances in quality and quantity of crops produced. Increased yield and advantageous combinations of desirable characteristics, such as resistance to disease and insects, heat and drought tolerance, and variations in plant composition, are all possible because of hybridization procedures. These procedures frequently rely heavily on providing for a male parent contributing pollen to a female parent to produce the resulting hybrid.

Field crops are bred through techniques that take advantage of the plant's method of pollination. A plant is self-pollinating if pollen from one flower is transferred to the same or another flower of the same plant or a genetically identical plant. A plant is cross-pollinated if the pollen comes from a flower on a genetically different plant.

In Brassica, the plant is normally self-sterile and can only be cross-pollinated. In self-pollinating species, such as soybeans and cotton, the male and female plants are anatomically juxtaposed. During natural pollination, the male reproductive organs of a given flower pollinate the female reproductive organs of the same flower.

Maize plants (Zea mays L.) are unusual in that they can be readily bred by both self-pollination and cross-pollination techniques. Maize has male flowers, located on the tassel, and female flowers, located on the ear, on the same plant. It can self-pollinate or cross-pollinate. Natural pollination occurs in maize when wind or gravity moves pollen from the tassels to the silks that protrude from the tops of the incipient ears.

A reliable method of controlling fertility in plants would offer the opportunity for improved plant breeding. This is especially true for development of maize hybrids, which typically relies upon some sort of male sterility system.

The development of maize hybrids requires the development of homozygous inbred lines, the crossing of these lines and the evaluation of the crosses. Pedigree breeding and recurrent selection are two of the breeding methods used to develop inbred lines from populations. Breeding programs combine desirable traits from two or more inbred lines or various broad-based sources into breeding pools from which new inbred lines are developed by selfing and selection of desired phenotypes. A hybrid maize variety is the cross of two such inbred lines, each of which may have one or more desirable characteristics lacked by the other or which complement the other. The new inbreds are crossed with other inbred lines and the hybrids from these crosses are evaluated to determine which have commercial potential. The hybrid progeny of the first generation is designated F₁. In the development of hybrids only the F₁ hybrid plants are sought. The F₁ hybrid is more vigorous than its inbred parents. This hybrid vigor, or heterosis, can be manifested in many ways, including increased vegetative growth and increased yield.

Hybrid maize seed can be produced by a male sterility system incorporating manual detasseling. To produce hybrid seed, the male tassel is removed from the growing female inbred parent, which can be planted in various alternating row patterns with the male inbred parent. Consequently, providing that there is sufficient isolation from sources of foreign maize pollen, the ears of the female inbred will be fertilized only with pollen from the male inbred. The resulting seed is therefore hybrid (F₁) and will form hybrid plants.

Environmental variation in plant development can result in plants tasseling after manual detasseling of the female parent is completed. Or, a detasseler might not completely remove the tassel of a female inbred plant. In any event, the result is that the female plant will successfully shed pollen and some female plants will be self-pollinated. This will result in seed of the female inbred being harvested along with the hybrid seed which is normally produced, a disadvantage to the grower because female inbred seed is not as productive as F₁ seed. In addition, the presence of female inbred seed can represent a germplasm security risk for the company producing the hybrid.

Alternatively, the female inbred can be mechanically detasseled. Mechanical detasseling is approximately as reliable as hand detasseling, but is faster and less costly. However, most detasseling machines produce more damage to the plants than does hand detasseling. Thus, no form of detasseling is presently entirely satisfactory and a need continues to exist for alternatives which further reduce production costs and eliminate self-pollination of the female parent in the production of hybrid seed.

A reliable system of genetic male sterility would provide advantages. The laborious detasseling process can be avoided in some genotypes by using cytoplasmic male-sterile (CMS) inbreds. In the absence of a fertility restorer gene, plants of a CMS inbred are male sterile as a result of factors resulting from the cytoplasmic, as opposed to the nuclear, genome. Thus, this characteristic is inherited exclusively through the female parent in maize plants, since only the female provides cytoplasm to the fertilized seed. CMS plants are fertilized with pollen from another inbred that is not male-sterile. Pollen from the second inbred may or may not contribute genes that make the hybrid plants male-fertile. Usually seed from detasseled normal maize and CMS produced seed of the same hybrid must be blended to insure that adequate pollen loads are available for fertilization when the hybrid plants are grown and to insure cytoplasmic diversity.

There can be other drawbacks to CMS. One is an historically observed association of a specific variant of CMS with susceptibility to certain crop diseases. This problem has discouraged widespread use of that CMS variant in producing hybrid maize and has had a negative impact on the use of CMS in maize in general.

In a number of circumstances, a male sterility plant trait is expressed by maintenance of a homozygous recessive condition. Difficulties arise in maintaining the homozygous condition, when a restoration gene must be used for maintenance. For example, a natural mutation in a gene critical to male sterility can impart a male sterility phenotype to plants when this mutant allele is in the homozygous state. This sterility can be restored when the non-mutant form of the gene is introduced into the plant. However, this form of restoration removes the desired homozygous recessive condition, restores full male fertility and prevents maintenance of pure male sterile maternal lines. This issue can be avoided where production of pollen containing the restoration gene is eliminated, thus providing a maintainer plant producing only pollen not containing the restoration gene and therefore the progeny retain the homozygous recessive condition.

As noted, an essential aspect of much of the work underway with male sterility systems is the identification of genes impacting male fertility. Such a gene can be used in a variety of systems to control male fertility including those described herein.

### SUMMARY OF THE INVENTION

This invention relates to nucleic acid sequences, and, specifically, DNA molecules and the amino acids encoded by the DNA molecules, which are critical to male fertility. A promoter of the DNA is identified. The invention also relates to use of such DNA molecules to mediate fertility in plants.

In the present invention the inventors provide novel DNA molecules and the amino acid sequence encoded that are critical to male fertility in plants. These can be used in any of the systems where control of fertility is useful, including those described above.

Thus, one object of the invention is to provide a nucleic acid sequence, the expression of which is critical to male fertility in plants.

Another object of the invention is to provide a DNA molecule encoding an amino acid sequence, the expression of which is critical to male fertility in plants.

A further object of the invention is to provide a method of using such DNA molecules to mediate male fertility in plants.

Further objects of the invention will become apparent in the description and claims that follow.

### BRIEF DESCRIPTION OF THE DRAWINGS

FIG. 1—Diagram of the chromosome 1 genomic region identified through map-based cloning techniques. Two genes were found in this interval: a predicted gene without any known homology, and a second gene with homology to plant R2-R3 myb proteins. The unknown gene was found to comprise a recombination, whereas the myb gene was flanked by recombinants and represented the candidate gene for Ms9.

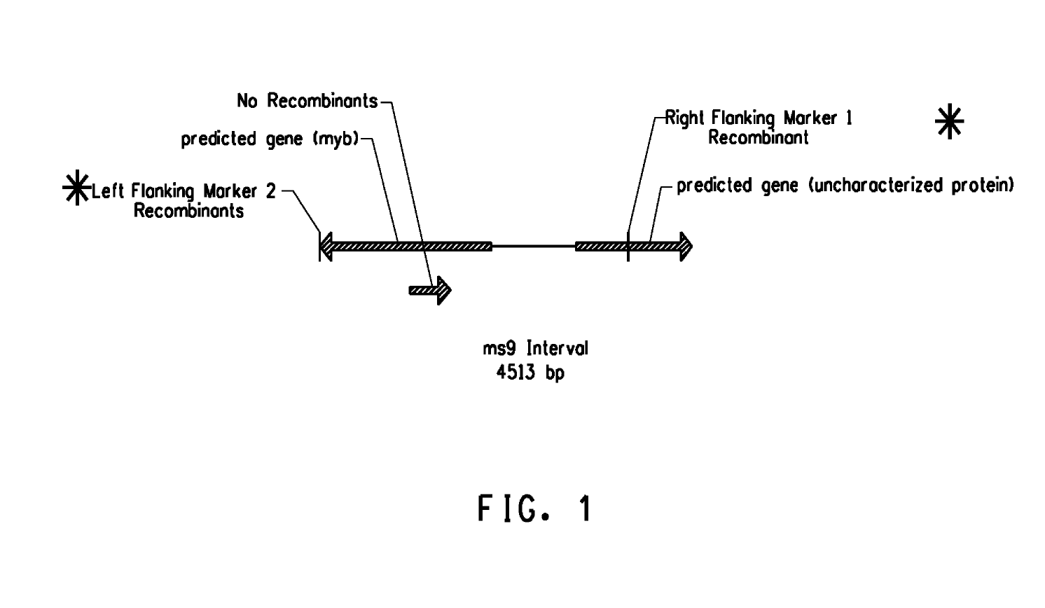

FIG. 2—Diagram of the native (wild-type) Ms9 gene showing intron and exon structure and alignment of portions of the native gene with exon1 of the ms9 reference allele and exon3 of the ms9-AD62A allele. The ms9-reference allele comparison aligns a portion of the ms9 reference allele CDS (SEQ ID NO: 6) with a portion of the ZmMS9 wild-type CDS (SEQ ID NO: 2). The ms9-AD62A allele comparison aligns a portion of the ms9-AD62A CDS (SEQ ID NO: 7) with a portion of the ZmMS9 wild-type CDS (SEQ ID NO: 2).

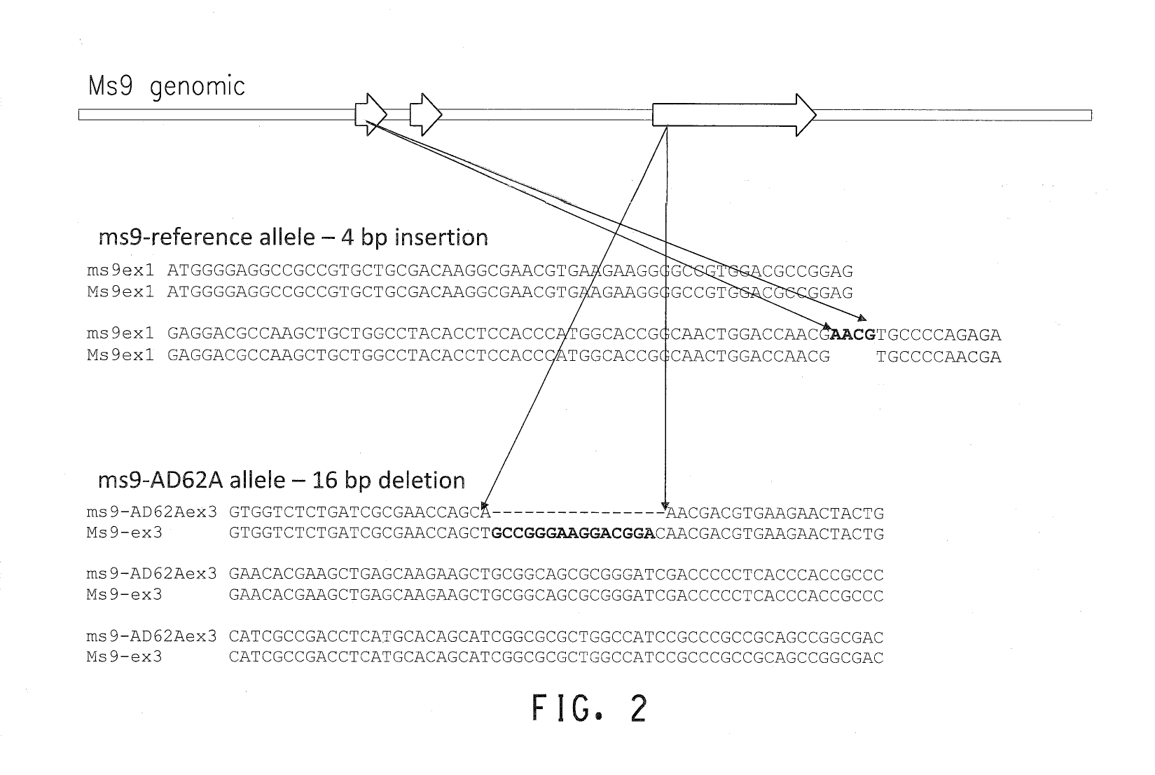

FIG. 3—Alignment of the protein translations for the Ms9 wild-type allele (SEQ ID NO: 3), the ms9 reference allele (SEQ ID NO: 10), and ms9-AD62A allele (SEQ ID NO: 9). The myb R2 domain is denoted by the black line above the alignment and the myb R3 domain is denoted by the black line below the alignment which the ms9-ref and ms9-AD62A mutations disrupt, respectively.

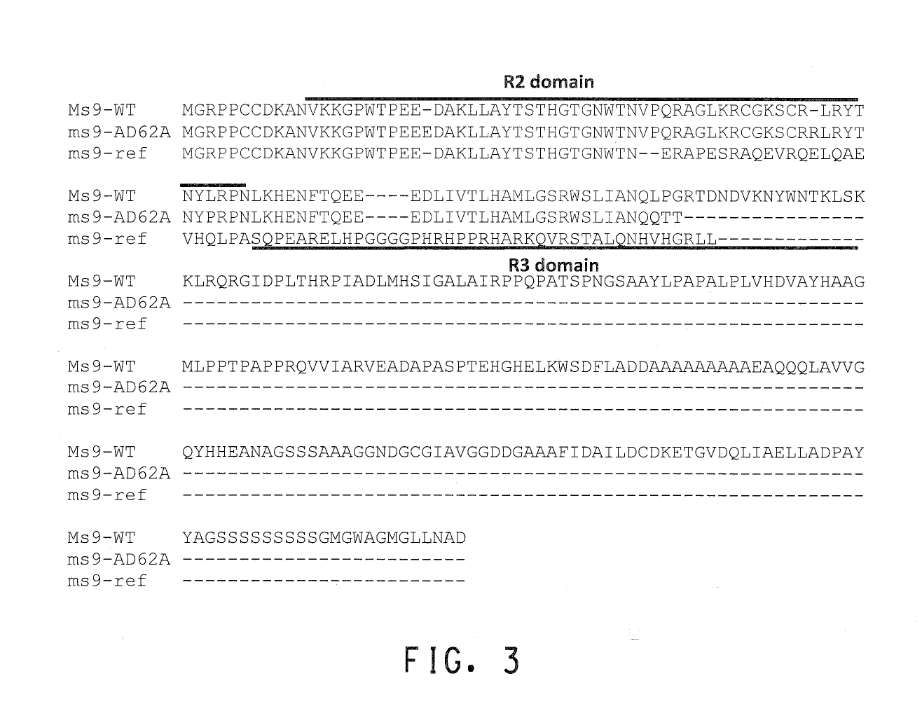

FIG. 4—Alignment of the Ms9 protein sequence from maize (SEQ ID NO: 3), sorghum (SEQ ID NO: 13), and rice (SEQ ID NO: 14).

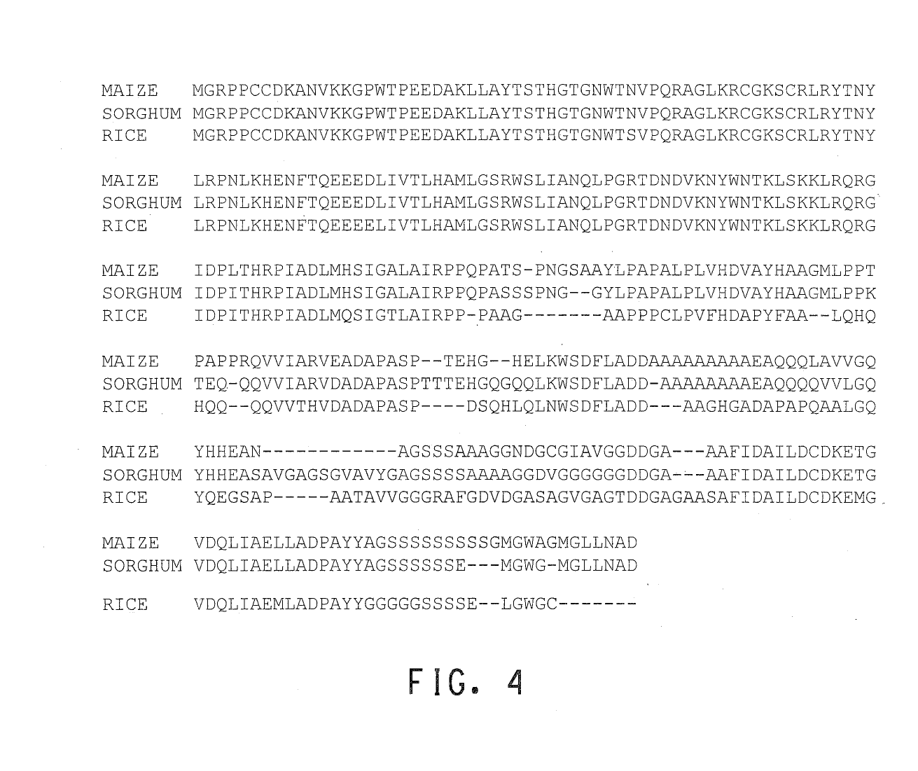

### BRIEF DESCRIPTION OF THE SEQUENCES

TABLE 1

| SEQ | PN/PP | | |
|---|---|---|---|
| ID | (Polynucleotide/ | | |
| NO: | Polypeptide) | ID | Species |
| 1 | PN | ZmMs9_wt_genomic | Zea mays |
| 2 | PN | ZmMs9_wt_CDS | Zea mays |
| 3 | PP | ZmMs9_wt_protein | Zea mays |
| 4 | PN | ZmMs9_sterile_reference_genomic | Zea mays |
| 5 | PN | Zmms9-AD62A_genomic | Zea mays |
| 6 | PN | Zmms9-cDS-exon1_insertion | Zea mays |
| 7 | PN | Zmms9-cDS-exon3_deletion | Zea mays |
| 8 | PN | Zmms9-cDS-_exon1_insertion_and | Zea mays |
| | | exon3_deletion | |
| 9 | PP | Zmms-FIG3 MS9-AD62A; PRT | Zea mays |
| 10 | PP | ms9-ref exon1 insertion protein | Zea mays |
| 11 | PP | ms9-AD62A exon3 protein | Zea mays |
| 12 | PP | ms9-exon1 insertion exon3 deletion protein | Zea mays |
| 13 | PP | sorghum MS9 protein | Sorghum bicolor |
| 14 | PP | rice MS9 protein | Oryza sativa |
| 15 | PN | ZmMs9-Promoter | Zea mays |

### DISCLOSURE OF THE INVENTION

All references referred to are incorporated herein by reference.

Unless defined otherwise, all technical and scientific terms used herein have the same meaning as commonly understood by one of ordinary skill in the art to which this invention belongs. Unless mentioned otherwise, the techniques employed or contemplated therein are standard methodologies well known to one of ordinary skill in the art. The materials, methods and examples are illustrative only and not limiting.

Genetic male sterility results from a mutation, suppression or other impact to one of the genes critical to a specific step in microsporogenesis, the term applied to the entire process of pollen formulation. These genes can be collectively referred to as male fertility genes (or, alternatively, male sterility genes). There are many steps in the overall pathway where gene function impacts fertility. This seems aptly supported by the frequency of genetic male sterility in maize. New alleles of male sterility mutants are uncovered in materials that range from elite inbreds to unadapted populations.

Thus the invention includes using the sequences shown herein to impact male fertility in a plant, that is, to control male fertility by manipulation of the genome using the genes of the invention. By way of example, without limitation, any of the methods described infra can be used with the sequence of the invention such as introducing a mutant sequence into a plant to cause sterility, causing mutation to the native sequence, introducing an antisense of the sequence into the plant, use of hairpin formations, linking it with other sequences to control its expression or any one of a myriad of processes available to one skilled in the art to impact male fertility in a plant.

The Ms9 phenotype was first identified in maize in 1932. Beadle, (1932) Genetics 17:413-431. It was found to be linked to the P1 gene on Chromosome 1. Breakdown of male reproductive tissue development occurs very early in premeiosis; tapetal cells may be affected as well. Greyson, et al., (1980) Can. J. Genet. Cytol. 22:153-166.

It will be evident to one skilled in the art that variations, mutations, derivations including fragments smaller than the entire sequence set forth may be used which retain the male sterility controlling properties of the gene. One of ordinary skill in the art can readily assess the variant or fragment by introduction into plants homozygous for a stable male sterile allele of Ms9, followed by observation of the plant's male tissue development.

The sequences of the invention may be isolated from any plant, including, but not limited to corn (Zea mays), canola (Brassica napus, Brassica rapa ssp.), alfalfa (Medicago sativa), rice (Oryza sativa), rye (Secale cereale), sorghum (Sorghum bicolor, Sorghum vulgare), sunflower (Helianthus annuus), wheat (Triticum aestivum), soybean (Glycine max), tobacco (Nicotiana tabacum), millet (Panicum spp.), potato (Solanum tuberosum), peanuts (Arachis hypogaea), cotton (Gossypium hirsutum), sweet potato (Ipomoea batatus), cassava (Manihot esculenta), coffee (Cofea spp.), coconut (Cocos nucifera), pineapple (Ananas comosus), citrus trees (Citrus spp.), cocoa (Theobroma cacao), tea (Camellia sinensis), banana (Musa spp.), avocado (Persea americana), fig (Ficus casica), guava (Psidium guajava), mango (Mangifera indica), olive (Olea europaea), oats (Avena sativa), barley (Hordeum vulgare), vegetables, ornamentals, and conifers. Preferably, plants include corn, soybean, sunflower, safflower, canola, wheat, barley, rye, alfalfa, rice, cotton and sorghum.

Sequences from other plants may be isolated according to well-known techniques based on their sequence homology to the homologous coding region of the coding sequences set forth herein. In these techniques, all or part of the known coding sequence is used as a probe which selectively hybridizes to other sequences present in a population of cloned genomic DNA fragments (i.e. genomic libraries) from a chosen organism. Methods are readily available in the art for the hybridization of nucleic acid sequences. An extensive guide to the hybridization of nucleic acids is found in Tijssen, Laboratory Techniques in Biochemistry and Molecular Biology—Hybridization with Nucleic Acid Probes, Part I, Chapter 2 “Overview of principles of hybridization and the strategy of nucleic acid probe assays”, Elsevier, New York (1993) and Current Protocols in Molecular Biology, Chapter 2, Ausubel, et al., Eds., Greene Publishing and Wiley-Interscience, New York (1995).

Thus the invention also includes those nucleotide sequences which selectively hybridize to the Ms9 nucleotide sequences under stringent conditions. In referring to a sequence that “selectively hybridizes” with Ms9, the term includes reference to hybridization, under stringent hybridization conditions, of a nucleic acid sequence to the specified nucleic acid target sequence to a detectably greater degree than its hybridization to non-target nucleic acid.

The terms “stringent conditions” or “stringent hybridization conditions” includes reference to conditions under which a probe will hybridize to its target sequence, to a detectably greater degree than to other sequences. Stringent conditions are target-sequence-dependent and will differ depending on the structure of the polynucleotide. By controlling the stringency of the hybridization and/or washing conditions, target sequences can be identified which are 100% complementary to a probe (homologous probing). Alternatively, stringency conditions can be adjusted to allow some mismatching in sequences so that lower degrees of similarity are detected (heterologous probing). Generally, probes of this type are in a range of about 1000 nucleotides in length to about 250 nucleotides in length.

An extensive guide to the hybridization of nucleic acids is found in Tijssen, Laboratory Techniques in Biochemistry and Molecular Biology—Hybridization with Nucleic Acid Probes, Part I, Chapter 2 “Overview of principles of hybridization and the strategy of nucleic acid probe assays”, Elsevier, New York (1993); and Current Protocols in Molecular Biology, Chapter 2, Ausubel, et al., Eds., Greene Publishing and Wiley-Interscience, New York (1995). See also, Sambrook, et al., (1989) Molecular Cloning: A Laboratory Manual (2nd ed. Cold Spring Harbor Laboratory, Cold Spring Harbor, N.Y.).

In general, sequences that correspond to the nucleotide sequences of the present invention and hybridize to the nucleotide sequence disclosed herein will be at least 50% homologous, 70% homologous, and even 85%, 86%, 87%, 88%, 89%, 90%, 91%, 92%, 93%, 94%, 95%, 96%, 97%, 98%, 99% homologous or more with the disclosed sequence. That is, the sequence similarity between probe and target may range, sharing at least about 50%, about 70% and even about 85% or more sequence similarity.

Specificity is typically the function of post-hybridization washes, the critical factors being the ionic strength and temperature of the final wash solution. Generally, stringent wash temperature conditions are selected to be about 5° C. to about 2° C. lower than the melting point (Tm) for the specific sequence at a defined ionic strength and pH. The melting point, or denaturation, of DNA occurs over a narrow temperature range and represents the disruption of the double helix into its complementary single strands. The process is described by the temperature of the midpoint of transition, Tm, which is also called the melting temperature. Formulas are available in the art for the determination of melting temperatures.

Preferred hybridization conditions for the nucleotide sequence of the invention include hybridization at 42° C. in 50% (w/v) formamide, 6×SSC, 0.5% (w/v) SDS, 100 (g/ml salmon sperm DNA. Exemplary low stringency washing conditions include hybridization at 42° C. in a solution of 2×SSC, 0.5% (w/v) SDS for 30 minutes and repeating. Exemplary moderate stringency conditions include a wash in 2×SSC, 0.5% (w/v) SDS at 50° C. for 30 minutes and repeating. Exemplary high stringency conditions include a wash in 0.1×SSC, 0.1% (w/v) SDS, at 65° C. for 30 minutes to one hour and repeating. Sequences that correspond to the promoter of the present invention may be obtained using all the above conditions. For purposes of defining the invention, the high stringency conditions are used.

The following terms are used to describe the sequence relationships between two or more nucleic acids or polynucleotides: (a) “reference sequence”, (b) “comparison window”, (c) “sequence identity” and (d) “percentage of sequence identity.”

(a) As used herein, “reference sequence” is a defined sequence used as a basis for sequence comparison. A reference sequence may be a subset or the entirety of a specified sequence; for example, as a segment of a full-length cDNA or gene sequence or the complete cDNA or gene sequence.

(b) As used herein, “comparison window” makes reference to a contiguous and specified segment of a polynucleotide sequence, wherein the polynucleotide sequence in the comparison window may comprise additions or deletions (i.e., gaps) compared to the reference sequence (which does not comprise additions or deletions) for optimal alignment of the two sequences. Generally, the comparison window is at least 20 contiguous nucleotides in length, and optionally can be 30, 40, 50 or 100 nucleotides in length or longer. Those of skill in the art understand that to avoid a high similarity to a reference sequence due to inclusion of gaps in the polynucleotide sequence a gap penalty is typically introduced and is subtracted from the number of matches.

Methods of aligning sequences for comparison are well-known in the art. Thus, the determination of percent sequence identity between any two sequences can be accomplished using a mathematical algorithm. Non-limiting examples of such mathematical algorithms are the algorithm of Myers and Miller, (1988) CABIOS 4:11-17; the local alignment algorithm of Smith, et al., (1981) Adv. Appl. Math. 2: 482; the global alignment algorithm of Needleman and Wunsch, (1970) J. Mol. Biol. 48: 443-453; the search-for-local-alignment-method of Pearson and Lipman, (1988) Proc. Natl. Acad. Sci. 85: 2444-2448; the algorithm of Karlin and Altschul, (1990) Proc. Natl. Acad. Sci. USA 87: 2264, modified as in Karlin and Altschul, (1993) Proc. Natl. Acad. Sci. USA 90: 5873-5877.

Computer implementations of these mathematical algorithms can be utilized for comparison of sequences to determine sequence identity. Such implementations include, but are not limited to: CLUSTAL in the PC/Gene program (available from Intelligenetics, Mountain View, Calif.); the ALIGN program (Version 2.0) and GAP, BESTFIT, BLAST, FASTA and TFASTA in the GCG Wisconsin Genetics Software Package, Version 10 (available from Accelrys Inc., 9685 Scranton Road, San Diego, Calif., USA). Alignments using these programs can be performed using the default parameters. The CLUSTAL program is well described by Higgins, et al., (1988) Gene 73:237-244 (1988); Higgins, et al., (1989) CABIOS 5:151-153; Corpet, et al., (1988) Nucleic Acids Res. 16:10881-90; Huang, et al., (1992) CABIOS 8:155-65 and Pearson, et al., (1994) Meth. Mol. Biol. 24:307-331. The ALIGN program is based on the algorithm of Myers and Miller, (1988) supra. A PAM120 weight residue table, a gap length penalty of 12 and a gap penalty of 4 can be used with the ALIGN program when comparing amino acid sequences. The BLAST programs of Altschul, et al., (1990) J. Mol. Biol. 215:403 are based on the algorithm of Karlin and Altschul, (1990) supra. BLAST nucleotide searches can be performed with the BLASTN program, score=100, wordlength=12, to obtain nucleotide sequences homologous to a nucleotide sequence encoding a protein of the invention. BLAST protein searches can be performed with the BLASTX program, score=50, wordlength=3, to obtain amino acid sequences homologous to a protein or polypeptide of the invention. To obtain gapped alignments for comparison purposes, Gapped BLAST (in BLAST 2.0) can be utilized as described in Altschul, et al., (1997) Nucleic Acids Res. 25:3389. Alternatively, PSI-BLAST (in BLAST 2.0) can be used to perform an iterated search that detects distant relationships between molecules. See, Altschul, et al., (1997) supra. When utilizing BLAST, Gapped BLAST, PSI-BLAST, the default parameters of the respective programs (e.g., BLASTN for nucleotide sequences, BLASTX for proteins) can be used. See http://www.ncbi.nlm.nih.gov. Alignment may also be performed manually by inspection.

Unless otherwise stated, sequence identity/similarity values provided herein refer to the value obtained using GAP Version 10 using the following parameters: % identity and % similarity for a nucleotide sequence using GAP Weight of 50 and Length Weight of 3 and the nwsgapdna.cmp scoring matrix; % identity and % similarity for an amino acid sequence using GAP Weight of 8 and Length Weight of 2 and the BLOSUM62 scoring matrix or any equivalent program thereof. By “equivalent program” is intended any sequence comparison program that, for any two sequences in question, generates an alignment having identical nucleotide or amino acid residue matches and an identical percent sequence identity when compared to the corresponding alignment generated by GAP Version 10.

GAP uses the algorithm of Needleman and Wunsch, (1970) J. Mol. Biol. 48:443-453, to find the alignment of two complete sequences that maximizes the number of matches and minimizes the number of gaps. GAP considers all possible alignments and gap positions and creates the alignment with the largest number of matched bases and the fewest gaps. It allows for the provision of a gap creation penalty and a gap extension penalty in units of matched bases. GAP must make a profit of gap creation penalty number of matches for each gap it inserts. If a gap extension penalty greater than zero is chosen, GAP must, in addition, make a profit for each gap inserted of the length of the gap times the gap extension penalty. Default gap creation penalty values and gap extension penalty values in Version 10 of the GCG Wisconsin Genetics Software Package for protein sequences are 8 and 2, respectively. For nucleotide sequences the default gap creation penalty is 50 while the default gap extension penalty is 3. The gap creation and gap extension penalties can be expressed as an integer selected from the group of integers consisting of from 0 to 200. Thus, for example, the gap creation and gap extension penalties can be 0, 1, 2, 3, 4, 5, 6, 7, 8, 9, 10, 15, 20, 25, 30, 35, 40, 45, 50, 55, 60, 65 or greater.

GAP presents one member of the family of best alignments. There may be many members of this family, but no other member has a better quality. GAP displays four figures of merit for alignments: Quality, Ratio, Identity and Similarity. The Quality is the metric maximized in order to align the sequences. Ratio is the quality divided by the number of bases in the shorter segment. Percent Identity is the percent of the symbols that actually match. Percent Similarity is the percent of the symbols that are similar. Symbols that are across from gaps are ignored. A similarity is scored when the scoring matrix value for a pair of symbols is greater than or equal to 0.50, the similarity threshold. The scoring matrix used in Version 10 of the GCG Wisconsin Genetics Software Package is BLOSUM62 (see, Henikoff and Henikoff, (1989) Proc. Natl. Acad. Sci. USA 89:10915).

(c) As used herein, “sequence identity” or “identity” in the context of two nucleic acid or polypeptide sequences makes reference to the residues in the two sequences that are the same when aligned for maximum correspondence over a specified comparison window. When percentage of sequence identity is used in reference to proteins it is recognized that residue positions which are not identical often differ by conservative amino acid substitutions, where amino acid residues are substituted for other amino acid residues with similar chemical properties (e.g., charge or hydrophobicity) and therefore do not change the functional properties of the molecule. When sequences differ in conservative substitutions, the percent sequence identity may be adjusted upwards to correct for the conservative nature of the substitution. Sequences that differ by such conservative substitutions are said to have “sequence similarity” or “similarity”. Means for making this adjustment are well known to those of skill in the art. Typically this involves scoring a conservative substitution as a partial rather than a full mismatch, thereby increasing the percentage sequence identity. Thus, for example, where an identical amino acid is given a score of 1 and a non-conservative substitution is given a score of zero, a conservative substitution is given a score between zero and 1. The scoring of conservative substitutions is calculated, e.g., as implemented in the program PC/GENE (Intelligenetics, Mountain View, Calif.).

(d) As used herein, “percentage of sequence identity” means the value determined by comparing two optimally aligned sequences over a comparison window, wherein the portion of the polynucleotide sequence in the comparison window may comprise additions or deletions (i.e., gaps) as compared to the reference sequence (which does not comprise additions or deletions) for optimal alignment of the two sequences. The percentage is calculated by determining the number of positions at which the identical nucleic acid base or amino acid residue occurs in both sequences to yield the number of matched positions, dividing the number of matched positions by the total number of positions in the window of comparison and multiplying the result by 100 to yield the percentage of sequence identity.

The use of the term “polynucleotide” is not intended to limit the present invention to polynucleotides comprising DNA. Those of ordinary skill in the art will recognize that polynucleotides can comprise ribonucleotides and combinations of ribonucleotides and deoxyribonucleotides. Such deoxyribonucleotides and ribonucleotides include both naturally occurring molecules and synthetic analogues. The polynucleotides of the invention also encompass all forms of sequences including, but not limited to, single-stranded forms, double-stranded forms, hairpins, stem-and-loop structures and the like.

Identity to the sequence of the present invention would mean a polynucleotide sequence having at least 65% sequence identity, more preferably at least 70% sequence identity, more preferably at least 75% sequence identity, more preferably at least 80% identity, more preferably at least 85% 86%, 87%, 88%, 89%, 90%, 91%, 92%, 93%, 94%, 95%, 96%, 97%, 98%, 99% sequence identity.

Promoter regions can be readily identified by one skilled in the art. The putative start codon containing the ATG motif is identified and upstream from the start codon is the presumptive promoter. By “promoter” is intended a regulatory region of DNA usually comprising a TATA box capable of directing RNA polymerase II to initiate RNA synthesis at the appropriate transcription initiation site for a particular coding sequence. A promoter can additionally comprise other recognition sequences generally positioned upstream or 5′ to the TATA box, referred to as upstream promoter elements, which influence the transcription initiation rate. It is recognized that having identified the nucleotide sequences for the promoter region disclosed herein, it is within the state of the art to isolate and identify further regulatory elements in the region upstream of the TATA box from the particular promoter region identified herein. Thus the promoter region disclosed herein is generally further defined by comprising upstream regulatory elements such as those responsible for tissue and temporal expression of the coding sequence, enhancers and the like. In the same manner, the promoter elements which enable expression in the desired tissue such as male tissue can be identified, isolated and used with other core promoters to confirm male tissue-preferred expression. By core promoter is meant the minimal sequence required to initiate transcription, such as the sequence called the TATA box which is common to promoters in genes encoding proteins. Thus the upstream promoter of Ms9 can optionally be used in conjunction with its own or core promoters from other sources. The promoter may be native or non-native to the cell in which it is found.

The isolated promoter sequence of the present invention can be modified to provide for a range of expression levels of the heterologous nucleotide sequence. Less than the entire promoter region can be utilized and the ability to drive anther-preferred expression retained. However, it is recognized that expression levels of mRNA can be decreased with deletions of portions of the promoter sequence. Thus, the promoter can be modified to be a weak or strong promoter. Generally, by “weak promoter” is intended a promoter that drives expression of a coding sequence at a low level. By “low level” is intended levels of about 1/10,000 transcripts to about 1/100,000 transcripts to about 1/500,000 transcripts. Conversely, a strong promoter drives expression of a coding sequence at a high level, or at about 1/10 transcripts to about 1/100 transcripts to about 1/1,000 transcripts. Generally, at least about 30 nucleotides of an isolated promoter sequence will be used to drive expression of a nucleotide sequence. It is recognized that to increase transcription levels, enhancers can be utilized in combination with the promoter regions of the invention. Enhancers are nucleotide sequences that act to increase the expression of a promoter region. Enhancers are known in the art and include the SV40 enhancer region, the 35S enhancer element, and the like.

The promoter of the present invention can be isolated from the 5′ region of its native coding region of 5′ untranslation region (5′UTR). Likewise the terminator can be isolated from the 3′ region flanking its respective stop codon. The term “isolated” refers to material such as a nucleic acid or protein which is substantially or essentially free from components which normally accompany or interact with the material as found in it naturally occurring environment or if the material is in its natural environment, the material has been altered by deliberate human intervention to a composition and/or placed at a locus in a cell other than the locus native to the material. Methods for isolation of promoter regions are well known in the art.

“Functional variants” of the regulatory sequences are also encompassed by the compositions of the present invention. Functional variants include, for example, the native regulatory sequences of the invention having one or more nucleotide substitutions, deletions or insertions. Functional variants of the invention may be created by site-directed mutagenesis, induced mutation or may occur as allelic variants (polymorphisms).

As used herein, a “functional fragment” of the regulatory sequence is a nucleotide sequence that is a regulatory sequence variant formed by one or more deletions from a larger sequence. For example, the 5′ portion of a promoter up to the TATA box near the transcription start site can be deleted without abolishing promoter activity, as described by Opsahl-Sorteberg, et al., (2004) Gene 341:49-58. Such variants should retain promoter activity, particularly the ability to drive expression in male tissues. Activity can be measured by Northern blot analysis, reporter activity measurements when using transcriptional fusions, and the like. See, for example, Sambrook, et al., (1989) Molecular Cloning: A Laboratory Manual (2nd ed. Cold Spring Harbor Laboratory, Cold Spring Harbor, N.Y.), herein incorporated by reference.

Functional fragments can be obtained by use of restriction enzymes to cleave the naturally occurring regulatory element nucleotide sequences disclosed herein; by synthesizing a nucleotide sequence from the naturally occurring DNA sequence; or can be obtained through the use of PCR technology. See particularly, Mullis, et al., (1987) Methods Enzymol. 155:335-350 and Erlich, ed. (1989) PCR Technology (Stockton Press, New York). Sequences which hybridize to the regulatory sequences of the present invention are within the scope of the invention. Sequences that correspond to the promoter sequences of the present invention and hybridize to the promoter sequences disclosed herein will be at least 50% homologous, 70% homologous, and even 85% 86%, 87%, 88%, 89%, 90%, 91%, 92%, 93%, 94%, 95%, 96%, 97%, 98%, 99% homologous or more with the disclosed sequence.

Smaller fragments may yet contain the regulatory properties of the promoter so identified and deletion analysis is one method of identifying essential regions. Deletion analysis can occur from both the 5′ and 3′ ends of the regulatory region. Fragments can be obtained by site-directed mutagenesis, mutagenesis using the polymerase chain reaction and the like. (See, Directed Mutagenesis: A Practical Approach IRL Press (1991)). The 3′ deletions can delineate the essential region and identify the 3′ end so that this region may then be operably linked to a core promoter of choice. Once the essential region is identified, transcription of an exogenous gene may be controlled by the essential region plus a core promoter. By core promoter is meant the sequence called the TATA box which is common to promoters in all genes encoding proteins. Thus the upstream promoter of Ms9 can optionally be used in conjunction with its own or core promoters from other sources. The promoter may be native or non-native to the cell in which it is found.

The core promoter can be any one of known core promoters such as the Cauliflower Mosaic Virus 35S or 19S promoter (U.S. Pat. No. 5,352,605), ubiquitin promoter (U.S. Pat. No. 5,510,474) the IN2 core promoter (U.S. Pat. No. 5,364,780) or a Figwort Mosaic Virus promoter (Gruber, et al., “Vectors for Plant Transformation” Methods in Plant Molecular Biology and Biotechnology et al. eds, CRC Press pp. 89-119 (1993)).

Promoter sequences from other plants may be isolated according to well-known techniques based on their sequence homology to the promoter sequence set forth herein. In these techniques, all or part of the known promoter sequence is used as a probe which selectively hybridizes to other sequences present in a population of cloned genomic DNA fragments (i.e., genomic libraries) from a chosen organism. Methods are readily available in the art for the hybridization of nucleic acid sequences.

The entire promoter sequence or portions thereof can be used as a probe capable of specifically hybridizing to corresponding promoter sequences. To achieve specific hybridization under a variety of conditions, such probes include sequences that are unique and are preferably at least about 10 nucleotides in length and most preferably at least about 20 nucleotides in length. Such probes can be used to amplify corresponding promoter sequences from a chosen organism by the well-known process of polymerase chain reaction (PCR). This technique can be used to isolate additional promoter sequences from a desired organism or as a diagnostic assay to determine the presence of the promoter sequence in an organism. Examples include hybridization screening of plated DNA libraries (either plaques or colonies; see, e.g., Innis, et al., eds., (1990) PCR Protocols, A Guide to Methods and Applications, Academic Press).

Further, a promoter of the present invention can be linked with nucleotide sequences other than the Ms9 gene to express other heterologous nucleotide sequences. The nucleotide sequence for the promoter of the invention, as well as fragments and variants thereof, can be provided in expression cassettes along with heterologous nucleotide sequences for expression in the plant of interest, more particularly in the male tissue of the plant. Such an expression cassette is provided with a plurality of restriction sites for insertion of the nucleotide sequence to be under the transcriptional regulation of the promoter. These expression cassettes are useful in the genetic manipulation of any plant to achieve a desired phenotypic response.

Phenotypic responses may be measured with respect to a control. A “control” or “control plant” or “control plant cell” provides a reference point for measuring changes in phenotype of a subject plant or plant cell in which genetic alteration, such as transformation, has been effected as to a gene of interest. A subject plant or plant cell may be descended from a plant or cell so altered and will comprise the alteration.

A control plant or plant cell may comprise, for example: (a) a wild-type (WT) plant or cell, i.e., of the same genotype as the starting material for the genetic alteration which resulted in the subject plant or cell; (b) a plant or plant cell of the same genotype as the starting material but which has been transformed with a null construct (i.e., with a construct which has no known effect on the trait of interest, such as a construct comprising a marker gene); (c) a plant or plant cell which is a non-transformed segregant among progeny of a subject plant or plant cell; (d) a plant or plant cell genetically identical to the subject plant or plant cell but which is not exposed to conditions or stimuli that would induce expression of the gene of interest or (e) the subject plant or plant cell itself, under conditions in which the gene of interest is not expressed. A control may comprise numerous individuals representing one or more of the categories above; for example, a collection of the non-transformed segregants of category “c” is often referred to as a bulk null.

Examples of other nucleotide sequences which can be used as the exogenous gene of the expression vector with the Ms9 promoter, or other promoters taught herein or known to those of skill in the art or other promoters taught herein or known to those of skill in the art complementary nucleotidic units such as antisense molecules (callase antisense RNA, barnase antisense RNA and chalcone synthase antisense RNA, Ms45 antisense RNA), ribozymes and external guide sequences, an aptamer or single stranded nucleotides. The exogenous nucleotide sequence can also encode carbohydrate degrading or modifying enzymes, amylases, debranching enzymes and pectinases, such as the alpha amylase gene disclosed in FIG. 24 of WO 2007/002267, auxins, rol B, cytotoxins, diptheria toxin, DAM methylase, avidin or may be selected from a prokaryotic regulatory system. By way of example, Mariani, et al., (1990) Nature 347:737, have shown that expression in the tapetum of either Aspergillus oryzae RNase-T1 or an RNase of Bacillus amyloliquefaciens, designated “barnase,” induced destruction of the tapetal cells, resulting in male infertility. Quaas, et al., (1988) Eur. J. Biochem. 173:617, describe the chemical synthesis of the RNase-T1, while the nucleotide sequence of the barnase gene is disclosed in Hartley, (1988) J. Molec. 202:913. The rolB gene of Agrobacterium rhizogenes codes for an enzyme that interferes with auxin metabolism by catalyzing the release of free indoles from indoxyl-β-glucosides. Estruch, et al., (1991) EMBO J. 11:3125 and Spena, et al., (1992) Theor. Appl. Genet. 84:520, have shown that the anther-specific expression of the rolB gene in tobacco resulted in plants having shriveled anthers in which pollen production was severely decreased and the rolB gene is an example of a gene that is useful for the control of pollen production. Slightom, et al., (1985) J. Biol. Chem. 261:108, disclose the nucleotide sequence of the rolB gene. DNA molecules encoding the diphtheria toxin gene can be obtained from the American Type Culture Collection (Rockville, Md.), ATCC Number 39359 or ATCC Number 67011 and see, Fabijanski, et al., EP Application Number 90902754.2, for examples and methods of use. The DAM methylase gene is used to cause sterility in the methods discussed at U.S. Pat. No. 5,689,049 and PCT/US95/15229 Cigan and Albertsen, “Reversible Nuclear Genetic System for Male Sterility in Transgenic Plants.” Also see, discussion of use of the avidin gene to cause sterility at U.S. Pat. No. 5,962,769 “Induction of Male Sterility in Plants by Expression of High Levels of Avidin” by Albertsen, et al.

The invention includes vectors with the Ms9 gene. A vector is prepared comprising Ms9, a promoter that will drive expression of the gene in the plant and a terminator region. As noted, the promoter in the construct may be the native promoter or a substituted promoter which will provide expression in the plant. The promoter in the construct may be an inducible promoter, so that expression of the sense or antisense molecule in the construct can be controlled by exposure to the inducer. In this regard, any plant-compatible promoter elements can be employed in the construct, influenced by the end result desired. Those can be plant gene promoters, such as, for example, the promoter for the small subunit of ribulose-1,5-bis-phosphate carboxylase or promoters from the tumor-inducing plasmids from Agrobacterium tumefaciens, such as the nopaline synthase and octopine synthase promoters or viral promoters such as the cauliflower mosaic virus (CaMV) 19S and 35S promoters or the figwort mosaic virus 35S promoter. See, Kay, et al., (1987) Science 236:1299 and EP Application Number 0 342 926; the barley lipid transfer protein promoter, LTP2 (Kalla, et al., (1994) Plant J. 6(6):849-60); the ubiquitin promoter (see, for example, U.S. Pat. No. 5,510,474); the END2 promoter (Linnestad, et al., U.S. Pat. No. 6,903,205) and the polygalacturonase PG47 promoter (see, Allen and Lonsdale, (1993) Plant J. 3:261-271; WO 1994/01572; U.S. Pat. No. 5,412,085). See, International Application Number WO 1991/19806 for a review of illustrative plant promoters suitably employed in the present invention.

The range of available plant compatible promoters includes tissue specific and inducible promoters. An inducible regulatory element is one that is capable of directly or indirectly activating transcription of one or more DNA sequences or genes in response to an inducer. In the absence of an inducer the DNA sequences or genes will not be transcribed. Typically the protein factor that binds specifically to an inducible regulatory element to activate transcription is present in an inactive form which is then directly or indirectly converted to the active form by the inducer. The inducer can be a chemical agent such as a protein, metabolite, growth regulator, herbicide or phenolic compound or a physiological stress imposed directly by heat, cold, salt or toxic elements or indirectly through the actin of a pathogen or disease agent such as a virus. A plant cell containing an inducible regulatory element may be exposed to an inducer by externally applying the inducer to the cell or plant such as by spraying, watering, heating or similar methods.

Any inducible promoter can be used in the instant invention. See, Ward, et al., (1993) Plant Mol. Biol. 22:361-366. Exemplary inducible promoters include ecdysone receptor promoters, U.S. Pat. No. 6,504,082; promoters from the ACE1 system which responds to copper (Mett, et al., (1993) PNAS 90:4567-4571); In2-1 and In2-2 gene from maize which respond to benzenesulfonamide herbicide safeners (U.S. Pat. No. 5,364,780; Hershey, et al., (1991) Mol. Gen. Genetics 227:229-237 and Gatz, et al., (1994) Mol. Gen. Genetics 243:32-38); the maize GST promoter, which is activated by hydrophobic electrophilic compounds that are used as pre-emergent herbicides and the tobacco PR-1a promoter, which is activated by salicylic acid. Other chemical-regulated promoters of interest include steroid-responsive promoters (see, for example, the glucocorticoid-inducible promoter in Schena, et al., (1991) Proc. Natl. Acad. Sci. USA 88:10421-10425 and McNellis, et al., (1998) Plant J. 14(2):247-257) and tetracycline-inducible and tetracycline-repressible promoters (see, for example, Gatz, et al., (1991) Mol. Gen. Genet. 227:229-237 and U.S. Pat. Nos. 5,814,618 and 5,789,156).

Tissue-preferred promoters can be utilized to target enhanced transcription and/or expression within a particular plant tissue. Promoters may express in the tissue of interest, along with expression in other plant tissue, may express strongly in the tissue of interest and to a much lesser degree than other tissue or may express highly preferably in the tissue of interest. Tissue-preferred promoters include those described in Yamamoto, et al., (1997) Plant J. 12(2):255-265; Kawamata, et al., (1997) Plant Cell Physiol. 38(7):792-803; Hansen, et al., (1997) Mol. Gen Genet. 254(3):337-343; Russell, et al., (1997) Transgenic Res. 6(2):157-168; Rinehart, et al., (1996) Plant Physiol. 112(3):1331-1341; Van Camp, et al., (1996) Plant Physiol. 112(2):525-535; Canevascini, et al., (1996) Plant Physiol. 112(2):513-524; Yamamoto, et al., (1994) Plant Cell Physiol. 35(5):773-778; Lam, (1994) Results Probl. Cell Differ. 20:181-196; Orozco, et al., (1993) Plant Mol Biol. 23(6):1129-1138; Matsuoka, et al., (1993) Proc Natl. Acad. Sci. USA 90(20):9586-9590 and Guevara-Garcia, et al., (1993) Plant J. 4(3):495-505. In one embodiment, the promoters are those which preferentially express to the male or female tissue of the plant. The invention does not require that any particular male tissue-preferred promoter be used in the process, and any of the many such promoters known to one skilled in the art may be employed. The native Ms9 promoter described herein is one example of a useful promoter. Another such promoter is the 5126 promoter, which preferentially directs expression of the gene to which it is linked to male tissue of the plants, as described in U.S. Pat. Nos. 5,837,851 and 5,689,051. Other examples include the Ms45 promoter described at U.S. Pat. No. 6,037,523; SF3 promoter described at U.S. Pat. No. 6,452,069; the BS92-7 promoter described at WO 2002/063021; a SGB6 regulatory element described at U.S. Pat. No. 5,470,359; the TA29 promoter (Koltunow, et al., (1990) Plant Cell 2:1201-1224; Goldberg, et al., (1993) Plant Cell 5:1217-1229 and U.S. Pat. No. 6,399,856); the type 2 metallothionein-like gene promoter (Charbonnel-Campaa, et al., Gene (2000) 254:199-208) and the Brassica Bca9 promoter (Lee, et al., (2003) Plant Cell Rep. 22:268-273).

Male gamete preferred promoters include the PG47 promoter, supra as well as ZM13 promoter (Hamilton, et al., (1998) Plant Mol. Biol. 38:663-669); actin depolymerizing factor promoters (such as Zmabp1, Zmabp2; see, for example Lopez, et al., (1996) Proc. Natl. Acad. Sci. USA 93:7415-7420); the promoter of the maize petctin methylesterase-liked gene, ZmC5 (Wakeley, et al., (1998) Plant Mol. Biol. 37:187-192); the profiling gene promoter Zmprol (Kovar, et al., (2000) The Plant Cell 12:583-598); the sulphated pentapeptide phytosulphokine gene ZmPSK1 (Lorbiecke, et al., (2005) Journal of Experimental Botany 56(417):1805-1819); the promoter of the calmodulin binding protein Mpcbp (Reddy, et al., (2000) J. Biol. Chem. 275(45):35457-70).

Other components of the vector may be included, also depending upon intended use of the gene. Examples include selectable markers, targeting or regulatory sequences, stabilizing or leader sequences, introns etc. General descriptions and examples of plant expression vectors and reporter genes can be found in Gruber, et al., “Vectors for Plant Transformation” in Method in Plant Molecular Biology and Biotechnology, Glick, et al., eds; CRC Press pp. 89-119 (1993). The selection of an appropriate expression vector will depend upon the host and the method of introducing the expression vector into the host. The expression cassette will also include at the 3′ terminus of the heterologous nucleotide sequence of interest, a transcriptional and translational termination region functional in plants. The termination region can be native with the promoter nucleotide sequence of the present invention, can be native with the DNA sequence of interest, or can be derived from another source. Convenient termination regions are available from the Ti-plasmid of A. tumefaciens, such as the octopine synthase and nopaline synthase termination regions. See also, Guerineau, et al., (1991) Mol. Gen. Genet. 262:141-144; Proudfoot, (1991) Cell 64:671-674; Sanfacon, et al., (1991) Genes Dev. 5:141-149; Mogen, et al., (1990) Plant Cell 2:1261-1272; Munroe, et al., (1990) Gene 91:151-158; Ballas, et al., (1989) Nucleic Acids Res. 17:7891-7903; Joshi, et al., (1987) Nucleic Acid Res. 15:9627-9639.

The expression cassettes can additionally contain 5′ leader sequences. Such leader sequences can act to enhance translation. Translation leaders are known in the art and include by way of example, picornavirus leaders, EMCV leader (Encephalomyocarditis 5′ noncoding region), Elroy-Stein, et al., (1989) Proc. Nat. Acad. Sci. USA 86:6126-6130; potyvirus leaders, for example, TEV leader (Tobacco Etch Virus), Allison, et al.; MDMV leader (Maize Dwarf Mosaic Virus), Virology 154:9-20 (1986); human immunoglobulin heavy-chain binding protein (BiP), Macejak, et al., (1991) Nature 353:90-94; untranslated leader from the coat protein mRNA of alfalfa mosaic virus (AMV RNA 4), Jobling, et al., (1987) Nature 325:622-625; Tobacco mosaic virus leader (TMV), Gallie, et al., (1989) Molecular Biology of RNA, pages 237-256 and maize chlorotic mottle virus leader (MCMV) Lommel, et al., (1991) Virology 81:382-385. See also, Della-Cioppa, et al., (1987) Plant Physiology 84:965-968. The cassette can also contain sequences that enhance translation and/or mRNA stability such as introns.

In those instances where it is desirable to have the expressed product of the heterologous nucleotide sequence directed to a particular organelle, particularly the plastid, amyloplast or to the endoplasmic reticulum or secreted at the cell's surface or extracellularly, the expression cassette can further comprise a coding sequence for a transit peptide. Such transit peptides are well known in the art and include, but are not limited to, the transit peptide for the acyl carrier protein, the small subunit of RUBISCO, plant EPSP synthase, Zea mays Brittle-1 chloroplast transit peptide (Nelson, et al., (1998) Plant Physiol 117(4):1235-1252; Sullivan, et al., Plant Cell 3(12):1337-48; Sullivan, et al., (1995) Planta 196(3):477-84; Sullivan, et al., (1992) J. Biol. Chem. 267(26):18999-9004) and the like. One skilled in the art will readily appreciate the many options available in expressing a product to a particular organelle. For example, the barley alpha amylase sequence is often used to direct expression to the endoplasmic reticulum (Rogers, (1985) J. Biol. Chem. 260:3731-3738). Use of transit peptides is well known (e.g., see, U.S. Pat. Nos. 5,717,084; 5,728,925).

In preparing the expression cassette, the various DNA fragments can be manipulated, so as to provide for the DNA sequences in the proper orientation and, as appropriate, in the proper reading frame. Toward this end, adapters or linkers can be employed to join the DNA fragments or other manipulations can be involved to provide for convenient restriction sites, removal of superfluous DNA, removal of restriction sites or the like. For this purpose, in vitro mutagenesis, primer repair, restriction digests, annealing and resubstitutions, such as transitions and transversions, can be involved.

As noted herein, the present invention provides vectors capable of expressing genes of interest. In general, the vectors should be functional in plant cells. At times, it may be preferable to have vectors that are functional in E. coli (e.g., production of protein for raising antibodies, DNA sequence analysis, construction of inserts, obtaining quantities of nucleic acids). Vectors and procedures for cloning and expression in E. coli are discussed in Sambrook, et al. (supra).

The transformation vector comprising the promoter sequence of the present invention operably linked to a heterologous nucleotide sequence in an expression cassette, can also contain at least one additional nucleotide sequence for a gene to be cotransformed into the organism. Alternatively, the additional sequence(s) can be provided on another transformation vector.

The cassette can include 5′ and 3′ regulatory sequences operably linked to a male fertility polynucleotide as disclosed herein. “Operably linked” is intended to mean a functional linkage between two or more elements. For example, an operable linkage between a polynucleotide of interest and a regulatory sequence (e.g., a promoter) is a functional link that allows for expression of the polynucleotide of interest. Operably linked elements may be contiguous or non-contiguous. When used to refer to the joining of two protein coding regions, by operably linked is intended that the coding regions are in the same reading frame.

Reporter genes can be included in the transformation vectors. Examples of suitable reporter genes known in the art can be found in, for example, Jefferson, et al., (1991) in Plant Molecular Biology Manual, ed. Gelvin, et al., (Kluwer Academic Publishers), pp. 1-33; DeWet, et al., (1987) Mol. Cell. Biol. 7:725-737; Goff, et al., (1990) EMBO J. 9:2517-2522; Kain, et al., (1995) BioTechniques 19:650-655 and Chiu, et al., (1996) Current Biology 6:325-330.

Selectable reporter genes for selection of transformed cells or tissues can be included in the transformation vectors. These can include genes that confer antibiotic resistance or resistance to herbicides. Examples of suitable selectable marker genes include, but are not limited to, genes encoding resistance to chloramphenicol, Herrera Estrella, et al., (1983) EMBO J. 2:987-992; methotrexate, Herrera Estrella, et al., (1983) Nature 303:209-213; Meijer, et al., (1991) Plant Mol. Biol. 16:807-820; hygromycin, Waldron, et al., (1985) Plant Mol. Biol. 5:103-108, Zhijian, et al., (1995) Plant Science 108:219-227; streptomycin, Jones, et al., (1987) Mol. Gen. Genet. 210:86-91; spectinomycin, Bretagne-Sagnard, et al., (1996) Transgenic Res. 5:131-137; bleomycin, Hille, et al., (1990) Plant Mol. Biol. 7:171-176; sulfonamide, Guerineau, et al., (1990) Plant Mol. Biol. 15:127-136; bromoxynil, Stalker, et al., (1988) Science 242:419-423; glyphosate, Shaw, et al., (1986) Science 233:478-481 and phosphinothricin, DeBlock, et al., (1987) EMBO J. 6:2513-2518.

Scorable or screenable markers may also be employed, where presence of the sequence produces a measurable product. Examples include a β-glucuronidase, or uidA gene (GUS), which encodes an enzyme for which various chromogenic substrates are known (for example, U.S. Pat. Nos. 5,268,463 and 5,599,670); chloramphenicol acetyl transferase (Jefferson, et al., The EMBO Journal 6(13):3901-3907) and alkaline phosphatase. Other screenable markers include the anthocyanin/flavonoid genes in general (see discussion at Taylor and Briggs, (1990) The Plant Cell 2:115-127) including, for example, a R-locus gene, which encodes a product that regulates the production of anthocyanin pigments (red color) in plant tissues (Dellaporta, et al., in Chromosome Structure and Function, Kluwer Academic Publishers, Appels and Gustafson eds., pp. 263-282 (1988)); the genes which control biosynthesis of flavonoid pigments, such as the maize C1 gene (Kao, et al., (1996) Plant Cell 8:1171-1179; Scheffler, et al., (1994) Mol. Gen. Genet. 242:40-48) and maize C2 (Wienand, et al., (1986) Mol. Gen. Genet. 203:202-207); the B gene (Chandler, et al., (1989) Plant Cell 1:1175-1183), the p1 gene (Grotewold, et al., (1991) Proc. Natl. Acad. Sci USA 88:4587-4591; Grotewold, et al., (1993) Cell 76:543-553; Sidorenko, et al., (1999) Plant Mol. Biol. 39:11-19); the bronze locus genes (Ralston, et al., (1988) Genetics 119:185-197; Nash, et al., (1990) Plant Cell 2(11):1039-1049), among others. Yet further examples of suitable markers include the cyan fluorescent protein (CYP) gene (Bolte, et al., (2004) J. Cell Science 117: 943-54 and Kato, et al., (2002) Plant Physiol 129:913-42), the yellow fluorescent protein gene (PhiYFP™ from Evrogen; see, Bolte, et al., (2004) J. Cell Science 117:943-54); a lux gene, which encodes a luciferase, the presence of which may be detected using, for example, X-ray film, scintillation counting, fluorescent spectrophotometry, low-light video cameras, photon counting cameras or multiwell luminometry (Teeri, et al., (1989) EMBO J. 8:343); a green fluorescent protein (GFP) gene (Sheen, et al., (1995) Plant J. 8(5):777-84) and DsRed2 where plant cells transformed with the marker gene are red in color, and thus visually selectable (Dietrich, et al., (2002) Biotechniques 2(2):286-293). Additional examples include a p-lactamase gene (Sutcliffe, (1978) Proc. Nat'l. Acad. Sci. U.S.A. 75:3737), which encodes an enzyme for which various chromogenic substrates are known (e.g., PADAC, a chromogenic cephalosporin); a xylE gene (Zukowsky, et al., (1983) Proc. Nat'l. Acad. Sci. U.S.A. 80:1101), which encodes a catechol dioxygenase that can convert chromogenic catechols; an α-amylase gene (Ikuta, et al., (1990) Biotech. 8:241) and a tyrosinase gene (Katz, et al., (1983) J. Gen. Microbiol. 129:2703), which encodes an enzyme capable of oxidizing tyrosine to DOPA and dopaquinone, which in turn condenses to form the easily detectable compound melanin. Clearly, many such markers are available to one skilled in the art.

The method of transformation/transfection is not critical to the instant invention; various methods of transformation or transfection are currently available. As newer methods are available to transform crops or other host cells they may be directly applied. Accordingly, a wide variety of methods have been developed to insert a DNA sequence into the genome of a host cell to obtain the transcription or transcript and translation of the sequence to effect phenotypic changes in the organism. Thus, any method which provides for efficient transformation/transfection may be employed.

Methods for introducing expression vectors into plant tissue available to one skilled in the art are varied and will depend on the plant selected. Procedures for transforming a wide variety of plant species are well known and described throughout the literature. See, for example, Miki, et al., “Procedures for Introducing Foreign DNA into Plants” in Methods in Plant Molecular Biotechnology, supra; Klein, et al., (1992) Bio/Technology 10:268 (1992) and Weising, et al., (1988) Ann. Rev. Genet. 22:421-477. For example, the DNA construct may be introduced into the genomic DNA of the plant cell using techniques such as microprojectile-mediated delivery, Klein, et al., (1987) Nature 327:70-73; electroporation, Fromm, et al., (1985) Proc. Natl. Acad. Sci. 82:5824; polyethylene glycol (PEG) precipitation, Paszkowski, et al., (1984) EMBO J. 3:2717-2722; direct gene transfer WO 1985/01856 and EP Number 0 275 069; in vitro protoplast transformation, U.S. Pat. No. 4,684,611 and microinjection of plant cell protoplasts or embryogenic callus, Crossway, (1985) Mol. Gen. Genetics 202:179-185. Co-cultivation of plant tissue with Agrobacterium tumefaciens is another option, where the DNA constructs are placed into a binary vector system. See e.g., U.S. Pat. No. 5,591,616; Ishida, et al., (1996) Nature Biotechnology 14:745-750. The virulence functions of the Agrobacterium tumefaciens host will direct the insertion of the construct into the plant cell DNA when the cell is infected by the bacteria. See, for example, Horsch, et al., (1984) Science 233:496-498 and Fraley, et al., (1983) Proc. Natl. Acad. Sci. 80:4803.

Standard methods for transformation of canola are described at Moloney, et al., (1989) Plant Cell Reports 8:238-242. Corn transformation is described by Fromm, et al., (1990) Bio/Technology 8:833 and Gordon-Kamm, et al., supra. Agrobacterium is primarily used in dicots, but certain monocots such as maize can be transformed by Agrobacterium. See, supra and U.S. Pat. No. 5,550,318. Rice transformation is described by Hiei, et al., (1994) The Plant Journal 6(2):271-282; Christou, et al., (1992) Trends in Biotechnology 10:239 and Lee, et al., (1991) Proc. Nat'l Acad. Sci. USA 88:6389. Wheat can be transformed by techniques similar to those used for transforming corn or rice. Sorghum transformation is described at Casas, et al., supra and sorghum by Wan, et al., (1994) Plant Physicol. 104:37. Soybean transformation is described in a number of publications, including U.S. Pat. No. 5,015,580.

When referring to “introduction” of the nucleotide sequence into a plant, it is meant that this can occur by direct transformation methods, such as Agrobacterium transformation of plant tissue, microprojectile bombardment, electroporation or any one of many methods known to one skilled in the art or, it can occur by crossing a plant having the heterologous nucleotide sequence with another plant so that progeny have the nucleotide sequence incorporated into their genomes. Such breeding techniques are well known to one skilled in the art.

The plant breeding methods used herein are well known to one skilled in the art. For a discussion of plant breeding techniques, see, Poehlman, (1987) Breeding Field Crops AVI Publication Co., Westport Conn. Many of the plants which would be most preferred in this method are bred through techniques that take advantage of the plant's method of pollination.

Backcrossing methods may be used to introduce a gene into the plants. This technique has been used for decades to introduce traits into a plant. An example of a description of this and other plant breeding methodologies that are well known can be found in references such as Plant Breeding Methodology, edit. Neal Jensen, John Wiley & Sons, Inc. (1988). In a typical backcross protocol, the original variety of interest (recurrent parent) is crossed to a second variety (nonrecurrent parent) that carries the single gene of interest to be transferred. The resulting progeny from this cross are then crossed again to the recurrent parent and the process is repeated until a plant is obtained wherein essentially all of the desired morphological and physiological characteristics of the recurrent parent are recovered in the converted plant, in addition to the single transferred gene from the nonrecurrent parent.

In certain embodiments of the invention, it is desirable to maintain the male sterile homozygous recessive condition of a male sterile plant, when using a transgenic restoration approach, while decreasing the number of plants, plantings and steps needed for maintenance plant with such traits. Homozygosity is a genetic condition existing when identical alleles reside at corresponding loci on homologous chromosomes. Heterozygosity is a genetic condition existing when different alleles reside at corresponding loci on homologous chromosomes. Hemizygosity is a genetic condition existing when there is only one copy of a gene (or set of genes) with no allelic counterpart on the sister chromosome. In an embodiment, the homozygous recessive condition results in conferring on the plant a trait of interest, which can be any trait desired and which results from the recessive genotype, such as increased drought or cold tolerance, early maturity, changed oil or protein content or any of a multitude of the many traits of interest to plant breeders. In one embodiment, the homozygous recessive condition confers male sterility upon the plant. When the sequence which is the functional complement of the homozygous condition is introduced into the plant (that is, a sequence which, when introduced into and expressed in the plant having the homozygous recessive condition, restores the wild-type condition), fertility is restored by virtue of restoration of the wild-type fertile phenotype.

Maintenance of the homozygous recessive condition is achieved by introducing a restoration transgene construct into a plant that is linked to a sequence which interferes with the function or formation of male gametes of the plant to create a maintainer or donor plant. The restoring transgene, upon introduction into a plant that is homozygous recessive for the genetic trait, restores the genetic function of that trait, with the plant producing only viable pollen containing a copy of the recessive allele but does not contain the restoration transgene. The transgene is kept in the hemizygous state in the maintainer plant. By transgene, it is meant any nucleic acid sequence which is introduced into the genome of a cell by genetic engineering techniques. A transgene may be a native DNA sequence, or a heterologous DNA sequence (i.e., “foreign DNA”). The term native DNA sequence refers to a nucleotide sequence which is naturally found in the cell but that may have been modified from its original form. The pollen from the maintainer can be used to fertilize plants that are homozygous for the recessive trait, and the progeny will therefore retain their homozygous recessive condition. The maintainer plant containing the restoring transgene construct is propagated by self-fertilization, with the resulting seed used to produce further plants that are homozygous recessive plants and contain the restoring transgene construct.

The maintainer plant serves as a pollen donor to the plant having the homozygous recessive trait. The maintainer is optimally produced from a plant having the homozygous recessive trait and which also has nucleotide sequences introduced therein which would restore the trait created by the homozygous recessive alleles. Further, the restoration sequence is linked to nucleotide sequences which interfere with the function or formation of male gametes. The gene can operate to prevent formation of male gametes or prevent function of the male gametes by any of a variety of well-know modalities and is not limited to a particular methodology. By way of example but not limitation, this can include use of genes which express a product cytotoxic to male gametes (See for example, U.S. Pat. Nos. 5,792,853; 5,689,049; PCT/EP89/00495); inhibit product formation of another gene important to male gamete function or formation (see, U.S. Pat. Nos. 5,859,341; 6,297,426); combine with another gene product to produce a substance preventing gene formation or function (see, U.S. Pat. Nos. 6,162,964; 6,013,859; 6,281,348; 6,399,856; 6,248,935; 6,750,868; 5,792,853); are antisense to or cause co-suppression of a gene critical to male gamete function or formation (see, U.S. Pat. Nos. 6,184,439; 5,728,926; 6,191,343; 5,728,558; 5,741,684); interfere with expression through use of hairpin formations (Smith, et al., (2000) Nature 407:319-320; WO 1999/53050 and WO 1998/53083) or the like. Many nucleotide sequences are known which inhibit pollen formation or function and any sequences which accomplish this function will suffice. A discussion of genes which can impact proper development or function is included at U.S. Pat. No. 6,399,856 and includes dominant negative genes such as cytotoxin genes, methylase genes and growth-inhibiting genes. Dominant negative genes include diphtheria toxin A-chain gene (Czako and An (1991) Plant Physiol. 95:687-692 and Greenfield, et al., (1983) PNAS 80:6853, Palmiter, et al., (1987) Cell 50:435); cell cycle division mutants such as CDC in maize (Colasanti, et al., (1991) PNAS 88:3377-3381); the WT gene (Farmer, et al., (1994) Hum. Mol. Genet. 3:723-728) and P68 (Chen, et al., (1991) PNAS 88:315-319).

Further examples of so-called “cytotoxic” genes are discussed supra and can include, but are not limited to pectate lyase gene pelE, from Erwinia chrysanthermi (Kenn, et al., (1986) J. Bacteriol. 168:595); T-urf13 gene from cms-T maize mitochondrial genomes (Braun, et al., (1990) Plant Cell 2:153; Dewey, et al., (1987) PNAS 84:5374); CytA toxin gene from Bacillus thuringiensis Israeliensis that causes cell membrane disruption (McLean, et al., (1987) J. Bacteriol 169:1017, U.S. Pat. No. 4,918,006); DNAses, RNAses, (U.S. Pat. No. 5,633,441); proteases or a genes expressing anti-sense RNA. A suitable gene may also encode a protein involved in inhibiting pistil development, pollen stigma interactions, pollen tube growth or fertilization or a combination thereof. In addition genes that either interfere with the normal accumulation of starch in pollen or affect osmotic balance within pollen may also be suitable.

In an illustrative embodiment, the DAM-methylase gene is used, discussed supra and at U.S. Pat. Nos. 5,792,852 and 5,689,049, the expression product of which catalyzes methylation of adenine residues in the DNA of the plant. Methylated adenines will affect cell viability and will be found only in the tissues in which the DAM-methylase gene is expressed. In another embodiment, an α-amylase gene can be used with a male tissue-preferred promoter. During the initial germinating period of cereal seeds, the aleurone layer cells will synthesize α-amylase, which participates in hydrolyzing starch to form glucose and maltose, so as to provide the nutrients needed for the growth of the germ (Rogers and Milliman, (1984) J. Biol. Chem. 259(19):12234-12240; Rogers, (1985) J. Biol. Chem. 260:3731-3738). In an embodiment, the α-amylase gene used can be the Zea mays α-amylase-1 gene. Young, et al., Plant Physiol. 105(2):759-760 and GenBank Accession Numbers L25805, GI:426481). Sequences encoding α-amylase are not typically found in pollen cells and when expression is directed to male tissue, the result is a breakdown of the energy source for the pollen grains and repression of pollen development.

One skilled in this area readily appreciates the methods described herein are applicable to any other crops which have the potential to outcross. By way of example, but not limitation it can include maize, soybean, sorghum or any plant with the capacity to outcross.

Ordinarily, to produce more plants having the recessive condition, one might cross the recessive plant with another recessive plant. This may not be desirable for some recessive traits and may be impossible for recessive traits affecting reproductive development. Alternatively, one could cross the homozygous plant with a second plant having the restoration gene, but this requires further crossing to segregate away the restoring gene to once again reach the recessive phenotypic state. Instead, in one process the homozygous recessive condition can be maintained, while crossing it with the maintainer plant. This method can be used with any situation in which is it desired to continue the recessive condition. This results in a cost-effective system that is relatively easy to operate to maintain a population of homozygous recessive plants.

A sporophytic gene is one which operates independently of the gametes. When the homozygous recessive condition is one which produces male sterility by preventing male sporophyte development, the maintainer plant, of necessity, must contain a functional restoring transgene construct capable of complementing the mutation and rendering the homozygous recessive plant able to produce functional pollen. Linking this sporophytic restoration gene with a second functional nucleotide sequence which interferes with the function or formation of the male gametes of the plant results in a maintainer plant that produces pollen containing only the recessive allele of the sporophytic gene at its native locus due to the action of the second nucleotide sequence in interfering with pollen formation or function. This functional pollen fraction is non-transgenic with regard to the restoring transgene construct.

In a still further embodiment, a marker gene, as discussed supra, may be provided in the construct with the restoring transgene. By way of example without limitation, use of a herbicide resistant marker such as bar allows one to eliminate cells, or progeny thereof, not having the restoring transgene. In yet another example, when using a scorable marker, such as a red fluorescent marker, such as DsRed2, any inadvertent transmission of the transgene can also be detected visually, and such escapes can be eliminated from progeny. Clearly, many other variations in the restoring construct are available to one skilled in the art.

In an illustrative embodiment, a method of maintaining a homozygous recessive condition of a male sterile plant at a genetic locus is provided, in which is employed a first nucleotide sequence which is a gene critical to male fertility, a second nucleotide sequence which inhibits the function or formation of viable male gametes, an optional third nucleotide sequence which is operably linked to the first sequence and preferentially expresses the sequence in male plant cells, an optional fourth nucleotide sequence operably linked to a fourth nucleotide sequence, the fourth sequence directing expression to male gametes, and an optional fifth nucleotide sequence which is a selectable or scorable marker allowing for selection of plant cells.

See, U.S. Pat. Nos. 5,478,369; 5,850,014; 6,265,640; 5,824,524; 7,696,405; and 7,759,543. In both the inbred and hybrid production processes, it is highly desired to maintain this homozygous recessive condition. When sequences encoding the Ms9 gene are introduced into a plant having the homozygous ms9ms9 condition, male fertility results. By the method of the invention, a plant which is ms9ms9 homozygous recessive may have introduced into it a functional sporophytic Ms9 gene, and thus is male fertile. This gene can be linked to a gene which operates to render pollen containing the restoring transgene construct nonfunctional or prevents its formation or which produces a lethal product in pollen, linked to the promoter directing its expression to the male gametes, to produce a plant that only produces functional pollen containing ms9 without the restoring transgene construct.

An example is a construct which includes the Ms9 gene, linked with a 5126 promoter, a male tissue-preferred promoter (see, U.S. Pat. Nos. 5,750,868; 5,837,851; 5,792,853; 5,689,049 and 5,689,051) and further linked to the cytotoxic DAM methylase gene under control of the polygalacturonase promoter, PG47 promoter (see, U.S. Pat. Nos. 5,545,546 and 5,412,085) in a hemizygotic condition. Therefore the resulting plant produces pollen, but the only viable pollen results from the allele not containing the restoring Ms9/DAM methylase construct and thus contains only the ms9 gene. It can therefore be used as a pollinator to fertilize the homozygous recessive plant (ms9/ms9) and progeny produced will continue to be male sterile as a result of maintaining homozygosity for ms9. The progeny will also not contain the introduced restoring transgene construct.

In yet another restoring construct example, the Ms9 gene is linked with a 5126 promoter, and further linked to the Zea mays α-amylase gene under control of the male tissue-preferred PG47 promoter. The scorable marker used in an embodiment is DS-RED EXPRESS (Clontech).

A desirable result of the process of the invention is that the plant having the restorer nucleotide sequence may be self-fertilized to achieve the propagation of restorer plants. The pollen will not have the restoring transgene construct, but the construct will be contained in 50% of the ovules (the female gamete). The seed resulting from the self-fertilization can be planted, and selection made for the seed or progeny plants having the restoring transgene construct. The selection process can occur by any one of many known processes, the most common being where the restoration nucleotide sequence is linked to a marker gene. The marker can be scorable or selectable, and allows identification of seed, or those plants produced from the seed, having the restoration gene.

In an embodiment of the invention, it is possible to provide that the male gamete-tissue preferred promoter is inducible. Additional control is thus allowed in the process, where so desired, by providing that the plant having the restoration nucleotide sequences is constitutively male sterile. This type of male sterility is set forth the in U.S. Pat. No. 5,859,341. In order for the plant to become fertile, the inducing substance must be provided and the plant will become fertile. Again, when combined with the process of the invention as described supra, the only pollen produced will not contain the restoration nucleotide sequences.

Further detailed description is provided below by way of instruction and illustration and is not intended to limit the scope of the invention.

### Example 1

### Identification and Cloning of Ms9

A map-based cloning approach was used to isolate and clone the maize ms9 gene. A small population of about 450 individuals was used to identify genetically linked flanking markers. A large population of about 2500 individuals was grown. Recombinants were identified using the flanking markers identified from the small population and along with newly-designed markers, the physical interval on chromosome 1 around the ms9 gene was determined. The one candidate gene for ms9 in this interval was found to be an R2/R3 plant-specific myb transcription factor. Such transcription factors act in a variety of plant-specific processes, including secondary metabolism (e.g. phenylpropanoid and tryptophan biosynthesis; cell determination and development, e.g. glabrous1, Werewolf, and Asymmetrical Leaves 1; and environmental response, e.g. fungal stress and low oxygen conditions. See, for example, Zhang, et al., (2007) Plant J 52:528-538; and Zhu, et al., (2008) Plant J. 55:266-277.

### Example 2

### Identification and Cloning of Additional Ms9 Alleles

The reference allele ms9-ref was found to contain a 4 basepair insertion in the first exon, causing a translation frame shift mutation. This mutation occurs in the R2 binding domain. A second allele, ms9-AD62A has a 16 bp deletion in the third exon which disrupts the R3 binding domain.

One of skill in the art recognizes that slight sequence variation at or near the mutation sites may be observed across maize inbreds. This may be due to, for example, native polymorphism in the genome and/or transposon insertion followed by imperfect excision.

### Example 3

### Expression Analysis and cDNA Isolation

Northern analysis can be used to detect expression of genes characteristic of anther development at various states of microsporogenesis. Northern analysis is also a commonly used technique known to those skilled in the art and is similar to Southern analysis except that mRNA rather than DNA is isolated and placed on the gel. The RNA is then hybridzed with the labeled probe. Potter, et al., (1981) Proc. Nat. Acad. Sci. USA 78:6662-6666, Lechelt, et al., (1989) Mol. Gen. Genet. 219:225-234.

Ms9 is natively expressed at high levels in the anther and has little to no expression in any other tissues. Within the anther, it has highest expression during the pollen mother cell, meiosis, quartet, and early uninucleate stages of development. The maize male sterile ms9 mutation has physiological impacts very early in microspore development, prior to meiosis. The Ms9 gene may be a control point for the entry into meiosis. This gene, in wild-type and/or mutated form, can be used in methods to control reproductive development, such as a Seed Production Technology (SPT) system described in U.S. Pat. No. 7,759,543 or U.S. Pat. No. 7,696,405. Orthologs exist for this gene in rice and sorghum, and this gene may be highly conserved across monocot crops, providing opportunities for fertility mediation in multiple species.

For example, regulation of expression of Ms9 in an inbred maize plant has value in hybrid seed production. Maize is an annual plant. Production of high-quality hybrid seed for planting each year by farmers requires use of pollination control systems in generating and propagating the inbred parents, and in crossing the inbred parents to produce hybrid seed. Regulation of expression of Ms9 can be a component of such pollination control systems.

In crop plants which are normally highly self-pollinated, such as rice or wheat, dominant suppression of Ms9 may be useful in creating male-sterile lines. Systems for maintaining such lines, and for restoring fertility to such lines or their progeny, have been described; see, for example, international patent application PCT/US2014/023932.

### Example 4

### Identification of Promoter and its Essential Regions

The identified Ms9 promoter, and variants and fragments thereof, has use in driving expression of an operably-linked heterologous polynucleotide. As noted above, the native expression of the maize Ms9 gene is highly preferential to anther tissue; the Ms9 promoter may be used to provide anther-tissue-preferred expression of the operably-linked heterologous polynucleotide. As further noted above, native expression of Ms9 occurs during the pollen mother cell, meiosis, quartet, and early uninucleate stages of development; the Ms9 promoter may be used to target expression during these stages in methods to control meiosis in developing male gametes. Alternatively or additionally, the promoter may be used for expression of genes other than its native MS9 during this critical stage of development.

The promoter sequence, or portions thereof, may also be used in methods of downregulating expression of a native or transgenic polynucleotide in a plant, for example through promoter-inverted-repeat constructs which target the promoter operably linked to the polynucleotide to be downregulated. See, for example, international patent publication WO 2008/112970; Mette, et al., (2000) EMBO J 19:5194-5201); and international patent application PCT/US2014/023932. The polynucleotide downregulated by means of a promoter-inverted-repeat-construct may be native or heterologous with respect to the targeted promoter.

A putative TATA box can be identified by primer extension analysis as described in by Current Protocols in Molecular Biology, Ausubel, et al., eds; John Wiley and Sons, New York pp. 4.8.1-4.8.5 (1987).

Regulatory regions of anther genes, such as promoters, may be identified in genomic subclones using functional analysis, usually verified by the observation of reporter gene expression in anther tissue and a lower level or absence of reporter gene expression in non-anther tissue. The possibility of the regulatory regions residing “upstream” or 5′ ward of the translational start site can be tested by subcloning a DNA fragment that contains the upstream region into expression vectors for transient expression experiments. It is expected that smaller subgenomic fragments may contain the regions essential for male-tissue preferred expression. For example, the essential regions of the CaMV 19S and 35S promoters have been identified in relatively small fragments derived from larger genomic pieces as described in U.S. Pat. No. 5,352,605.

The selection of an appropriate expression vector with which to test for functional expression will depend upon the host and the method of introducing the expression vector into the host and such methods are well known to one skilled in the art. For eukaryotes, the regions in the vector include regions that control initiation of transcription and control processing. These regions are operably linked to a reporter gene such as UidA, encoding-glucuronidase (GUS), or luciferase. General descriptions and examples of plant expression vectors and reporter genes can be found in Gruber, et al., “Vectors for Plant Transformation” in Methods in Plant Molecular Biology and Biotechnology; Glick, et al., eds; CRC Press; pp. 89-119; (1993). GUS expression vectors and GUS gene cassettes are commercially available from Clonetech, Palo Alto, Calif., while luciferase expression vectors and luciferase gene cassettes are available from Promega Corporation, Madison, Wis. Ti plasmids and other Agrobacterium vectors are described in Ishida, et al., (1996) Nature Biotechnology 14:745-750 and in U.S. Pat. No. 5,591,616.

Expression vectors containing putative regulatory regions located in genomic fragments can be introduced into intact tissues such as staged anthers, embryos or into callus. Methods of DNA delivery include microprojectile bombardment, DNA injection, electroporation and Agrobacterium-mediated gene transfer (see, Gruber, et al., “Vectors for Plant Transformation,” in Methods in Plant Molecular Biology and Biotechnology, Glick, et al., eds.; CRC Press; (1993); U.S. Pat. No. 5,591,616 and Ishida, et al., (1996) Nature Biotechnology 14:745-750). General methods of culturing plant tissues are found in Gruber, et al., supra and Glick, supra.

For the transient assay system, staged, isolated anthers are immediately placed onto tassel culture medium (Pareddy and Petelino, (1989) Crop Sci. J.; 29:1564-1566) solidified with 0.5% Phytagel (Sigma, St. Louis) or other solidifying media. The expression vector DNA is introduced within 5 hours preferably by microprojectile-mediated delivery with 1.2 μm particles at 1000-1100 Psi. After DNA delivery, the anthers are incubated at 26° C. upon the same tassel culture medium for 17 hours and analyzed by preparing a whole tissue homogenate and assaying for GUS or for lucifierase activity (see, Gruber, et al., supra).

Deletion analysis can occur from both the 5′ and 3′ ends of the regulatory region: fragments can be obtained by site-directed mutagenesis, mutagenesis using the polymerase chain reaction, and the like (Directed Mutagenesis: A Practical Approach; IRL Press; (1991)). The 3′ end of the male tissue-preferred regulatory region can be delineated by proximity to the putative TATA box or by 3′ deletions if necessary. The essential region may then be operably linked to a core promoter of choice. Once the essential region is identified, transcription of an exogenous gene may be controlled by the male tissue-preferred region of Ms9 plus a core promoter. The core promoter can be any one of known core promoters such as a Cauliflower Mosaic Virus 35S or 19S promoter (U.S. Pat. No. 5,352,605), Ubiquitin (U.S. Pat. No. 5,510,474), the IN2 core promoter (U.S. Pat. No. 5,364,780) or a Figwort Mosaic Virus promoter (Gruber, et al., “Vectors for Plant Transformation” in Methods in Plant Molecular Biology and Biotechnology; Glick, et al., eds.; CRC Press; pp. 89-119; (1993)). Preferably, the promoter is the core promoter of a male tissue-preferred gene or the CaMV 35S core promoter. More preferably, the promoter is a promoter of a male tissue-preferred gene and in particular, the Ms9 core promoter.

Further mutational analysis, for example by linker scanning, a method well known to the art, can identify small segments containing sequences required for anther-preferred expression. These mutations may introduce modifications of functionality such as in the levels of expression, in the timing of expression, or in the tissue of expression. Mutations may also be silent and have no observable effect.

What is claimed is:

1. A method of regulating plant male fertility, comprising controlling the expression of a polynucleotide in a plant, wherein the polynucleotide is selected from the group consisting of:
a polynucleotide encoding a polypeptide that is at least 90% identical to the full length of a polypeptide of SEQ ID NO: 13 or 14
wherein the polynucleotide encodes a polypeptide that impacts male fertility in a plant.

2. The method of claim 1 wherein the polypeptide is present at a lower level in said plant, compared to a control plant.

3. The method of claim 1 wherein the polypeptide is present at a higher level in said plant, compared to a control plant.

4. (canceled)

5. (canceled)

6. (canceled)

7. A method of regulating male fertility in a first plant species, comprising the steps of:
a) downregulating expression of the native MS9 gene in a plant of said first species so as to result in male sterility; and
b) introducing a construct comprising a polynucleotide encoding an MS9 polypeptide native to a second plant species,
wherein the introduced polynucleotide restores fertility to the plant of the first species or to progeny of the plant of the first species.

8. The method of claim 7, wherein said first species is selected from the group consisting of maize, sorghum, and rice; and wherein said second species is selected from the group consisting of maize, sorghum, and rice.

9. The method of claim 7 wherein said downregulation is achieved by targeting the promoter natively associated with the polynucleotide encoding the native MS9 polypeptide of the first species.

10. The method of claim 7 wherein said introducing is by crossing.

11. A method of maintaining a homozygous recessive condition of a male sterile plant, the method comprising:
(a) Providing a first plant comprising homozygous recessive alleles of the ms9 gene, wherein said plant is male-sterile;
(b) Introducing a construct into a second plant, the second plant comprising homozygous recessive alleles of the ms9 gene, the construct comprising:
(i) a first nucleotide sequence comprising the Ms9 nucleotide sequence, wherein said first sequence is selected from the group consisting of:
a. a sequence encoding a polypeptide selected from the group consisting of SEQ ID NO: 3, 13, and 14;
b. the sequence of SEQ ID NO: 1 or 2; and
c. a sequence having at least 90% identity to the full length of SEQ ID NO: 1 or 2;
which when expressed in the first plant would restore male fertility;
(ii) a second nucleotide sequence that when expressed inhibits the function or formation of male gametes in the second plant, such that functional male gametes produced by the second plant do not contain the construct; and
(iii) fertilizing the first plant with male gametes of the second plant to produce progeny which maintain the homozygous recessive condition of the first plant.

12.-27. (canceled)


SEQUENCE LISTING (15 sequences)

SEQ ID NO: 1 - DNA, 4366 bp, Zea mays
rccctcctcgccgtcggacgccatggccgcaccgacctcgccgccgctcccgccattggccccggacatcgttgcgccgatcttggacgccacagcgctcaggaacccgccctcctcagctggcttgccgccgccgccgccgccaacaccaccggcctccgctacttcttggcgcgcctttttgtgcggcatcgtctcgctctgctccctcctctcatccgcgcccaccgaatcctccatccggaaacgaaaccgctagagctaaggtctcaatcaacacagtgtcgagtgtggacaagttggtgctgctggactgctggcacatatacgaaaacgaaagcaggtcaggtggagagcagaggaggagccgtgccgcaggcagctattttatagctgctcgacgcgggtgctacacgcgtccagtgagaacggctgcctttctcggagcgcgtagcctggcgcgcgctggttggctccactctgcggcgttttttcaatttgtttttttactcctctttctgcggggaggcggcgagatgtcgggtggacggtggatcggcaaacgaagggcctgtcggtgtggacggtggagatgcactaagccttctttgagcaggtcacatcttttgcaagttcatggtgcccagtacgtagtggacagcagagaagctccgggcggggccaatgcaaaaccgatctggcggcgtcgatgtcgtgaaaaccgtcatgcccaactcgtgaaaattttaaaccccagcatcaccaggccgctagctccgtctctcaacgataagattacccacaccaacccgcgctcgccctaatgagctgttgatttgccggagggaagcagttgcgcgcgcgctctactatactgtcgccgccgccataaacaaagagggaaccagcgtctcttccctaatctaaccatctcctgcgtgattgacactaaccatgccgtggctagttaaatgacggggacggggtcacgccttcgttgcgtgcctccacctccccccctcggcgcccccaacgacatgttgttaccgtggctgtggcagccggccggtctccttctccatccatatgtactggcagcatcgtatcacctttttttctgcagcggtgatctcatctaggcgtcggtcagagctctctcgagctcgccagcggtggttggtcgtcgtcgtcgtcgtcgtcgatggggaggccgccgtgctgcgacaaggcgaacgtgaagaaggggccgtggacgccggaggaggacgccaagctgctggcctacacctccacccatggcaccggcaactggaccaacgtgccccaacgagcaggtgatcgtgccgccgtgcaccatgcattttgttgttttgtttgtggctcatgacgaggcgctggcggacggtgcggcatgatgtgatatgatgcgcagggctcaagaggtgcggcaagagctgcaggctgaggtacaccaactacctgcgtcccaacctgaagcacgagaacttcacccaggaggaggaagacctcatcgtcaccctccacgccatgctcggaagcaggtacgcagtacagcgctgcagaatcatgttcatggccgtttgctttgattaattccacacaacatgcatgcatgcattcgcatcatccttcagcttcctcacctaggaaccggatagatccttcgtagtgctgcagataaatccgatttatcttctatctcaatgcggttttgaaactaagtatatcacattagatagtgttaattgctgaactgaagaagatctgaattatgaaagacacgtccgatcctgcagctcttacaaaggttttctctttttttaagaaaaaaaatctgcctccatttaccgtaggtgattcttcctggacatttttgttccgcggcaaattaaatagtaattgaacctatgtttcacatgagaaaattgctagtaatcgggtgtttggaaatgattctgaatcttgcggacttaaatctgaaaccaatcgtcccaatgcaattcgctagagcaattgatctgttcatttccaatcagtcaatcaccaagccctagaaaacggacagctagttcagtagttcccgcatcagcgccattgctgatggatcgaacagctgacgcgaatgaaaacgacatgacaccgtcggggagatcgttggatgagttccgagcgataacgaactgtacgggcagtgacatacacaatgcgtgcgcgcatgcaaagttgattggaatccaatgcgtccagctgataggagtatttacactacagatacactcatagttgctagggtaggtgatcttgagatgcatcttgatccctcgctagttagtactattcatgctatttgctgcagttaattaacgggtccggcctgcaatggaaattgtagtgcgctagaccgcgcgctgctgatctgggccacgaactgcgcgcgtttgcatgcaggtggtctctgatcgcgaaccagctgccgggaaggacggacaacgacgtgaagaactactggaacacgaagctgagcaagaagctgcggcagcgcgggatcgaccccctcacccaccgccccatcgccgacctcatgcacagcatcggcgcgctggccatccgcccgccgcagccggcgacctcccctaacggctccgccgcctaccttcctgcgccggcgctcccgctcgtccacgacgtcgcgtaccacgccgccggaatgctgccgccgacgccggcgccgccccggcaggtcgtcatcgcgcgcgtggaagcggacgcgcccgcgtcgccgacggagcacgggcacgagctcaagtggagcgacttcctcgccgacgacgccgccgccgcggcggcggccgcggccgaggcgcagcagcagctggccgttgttgggcagtaccaccacgaggccaacgccgggagcagcagcgctgcggccggcggtaacgacggttgtggcattgccgtcggcggcgacgacggcgcagcggcgttcatcgacgccatcctggactgcgacaaggagacgggggtggaccagctcatcgccgagctgctggccgacccggcctactacgcgggctcctcctcctcctcctcctcctcgtccgggatgggctgggccggcatgggcctgctgaacgctgattaattaactcaagactgctttagtgtttgctatacgtacttaccatcaattagtatgatggtcaaaccttccaaccggatccattcatatgcttgcacaactctgggagtctgggtgttttcggattacaaattgtacggataattgacgccatttgtgcgtgtgtgtctcattcattttcctagaggaaactgtgtttgtgttgtgtggttcaagctgccgctggtataacttggcacgtctcacggcacctgaaaaaaaatcacgggcaggcttgcgtcgttgcatcggtcgccaccacacaccggccggcccctatcacccgtttcctcacgacggaacgggacgatgccgcagcagtcagcgtaacaaaaagaaagcaaagggtgaaaggggaagggggagaataatctcggtttttagcacgcaaacacacggcacgagcagctgagcgcccatggcaggtccccggcgtctcctactcctcgtcccgctgctcgtgctcctcggcgcgcacccgccgcagtgcgggtccgcggaggaggggacgaaggtctccctggagctctactacgagtcgttgtgcccgtactgctcgcggttcatcgtcaaccgcctcgcggggatcttcgaggacgggctgatcgacgccgtccacctccggctcgtcccctacggcaacgcgcgcgtcgcatccaacagcgagatctcttgccaggtactcctactaatttatcatgatagcagcgattcttccaatcatctagtactcctactaattcatcatgatagcagcgattcttcccatcatctcaaaactgtgttaaatgaatcgcctttcatcggttcgcttataatttgcttaagctagaaggttgtacacggtagcggtggtgtgagcgtggcatattagcgcccacaccatccggcatacttttagggctacacgaagcacatgtcttatttcacacggtggcgattatatcataaatgttttggtctgatctcataaaaatttacggttccttttaagtataactatgatggtgcgcgcaaccatcaaaagatttaagggtgtctaacccctacttttaatcactatagataacctagatcaaacgttaatattgtggtctaattgatctaagtgcttcataaaacacttacactaataattgagggattttgagcatttgggtgatt

SEQ ID NO: 2 - DNA, 959 bp, Zea mays
atggggaggccgccgtgctgcgacaaggcgaacgtgaagaaggggccgtggacgccggaggaggacgccaagctgctggcctacacctccacccatggcaccggcaactggaccaacgtgccccaacgagcagggctcaagaggtgcggcaagagctgcaggctgaggtacaccaactacctgcgtcccaacctgaagcacgagaacttcacccaggaggaggaagacctcatcgtcaccctccacgccatgctcggaagcaggtggtctctgatcgcgaaccagctgccgggaaggacggacaacgacgtgaagaactactggaacacgaagctgagcaagaagctgcggcagcgcgggatcgaccccctcacccaccgccccatcgccgacctcatgcacagcatcggcgcgctggccatccgcccgccgcagccggcgacctcccctaacggctccgccgcctaccttcctgcgccggcgctcccgctcgtccacgacgtcgcgtaccacgccgccggaatgctgccgccgacgccggcgccgccccggcaggtcgtcatcgcgcgcgtggaagcggacgcgcccgcgtcgccgacggagcacgggcacgagctcaagtggagcgacttcctcgccgacgacgccgccgccgcggcggcggccgcggccgaggcgcagcagcagctggccgttgttgggcagtaccaccacgaggccaacgccgggagcagcagcgctgcggccggcggtaacgacggttgtggcattgccgtcggcggcgacgacggcgcagcggcgttcatcgacgccatcctggactgcgacaaggagacgggggtggaccagctcatcgccgagctgctggccgacccggcctactacgcgggctcctcctcctcctcctcctcctcgtccgggatgggctgggccggcatgggcctgctgaacgctgatta

SEQ ID NO: 3 - protein, 319 aa, Zea mays
MGRPPCCDKANVKKGPWTPEEDAKLLAYTSTHGTGNWTNVPQRAGLKRCGKSCRLRYTNYLRPNLKHENFTQEEEDLIVTLHAMLGSRWSLIANQLPGRTDNDVKNYWNTKLSKKLRQRGIDPLTHRPIADLMHSIGALAIRPPQPATSPNGSAAYLPAPALPLVHDVAYHAAGMLPPTPAPPRQVVIARVEADAPASPTEHGHELKWSDFLADDAAAAAAAAAEAQQQLAVVGQYHHEANAGSSSAAAGGNDGCGIAVGGDDGAAAFIDAILDCDKETGVDQLIAELLADPAYYAGSSSSSSSSSGMGWAGMGLLNAD

SEQ ID NO: 4 - DNA, 2249 bp, Zea mays
tgacgggacggggtcacgccttcgtcgcgtgcctccacctccccccctcggcgcccccaacgacatgttgttaccgtggctgtggcagccggccggtctccttctccatccatatgtactggcagcatcgtatcacctttttctgcagcggtgatctcatctaggcgtcggtcagagctctctcgagctcgccagcggtggttggtcgtcgtcgtcgtcgtcgtcgatggggaggccgccgtgctgcgacaaggcgaacgtgaagaaggggccgtggacgccggaggaggacgccaagctgctggcctacacctccacccatggcaccggcaactggaccaacgaacgtgccccagagagcaggtgatcgtgccgccgtgcaccatgcattttgttgttttgtttgtggctcatgacgaggcgctggcggacggtgcggcatgatgtgatatgatgcgcagggctcaagaggtgcggcaagagctgcaggctgaggtacaccaactacctgcgtcccaacctgaagcacgagaacttcacccaggaggaggaggacctcatcgtcaccctccacgccatgctcggaagcaggtacgcagtacagcgctgcagaatcatgttcatggccgtttgctttgatttgtttccgcggctgctactcctttccacacaacatgcatgcattcgcatcatccttcagcttcctcacctaggaaccggatagatccttcgtagtgctgcagataaatccgatttatcttctatctcaatgcggttttgaaactaagtatatcacattagatagtgttaattgctgaactgaagaagatctgaattatgaaagacacgtccgatcctgcagctcttacaaaggttttctctttttttaagaaaaaaaaatcggcctccatttaccgtaggtgattcttcctggacatttttgttccgcggcaaattaaatagtaattgaacctatgtttcacatgagaaaattgctagtaatcgggtgtttggaaatgattctgaatcttgcggacttaaatctgaaaccaatcgtcccaatgcaatttgctagagcaattgatctgttcatttccaatcaccaagccctagaaacggacgggacagctagttcagtagttcccgcatcagcgccattgctgatggatcgaacagctgacgcgaatgaaaacgacatgacaccgtcggggagatcgttggatgagttccgagcgataacgaactgtacgggcagtgacatacacaatgcgtgcgcgcatgcaaagttgattggaatccatgcgtccagctgatcactcgtagtcgtagtcatagttgctagggtaggtgatcttgagatgcatcttgatccctcgctagttagatcatgctatttgctgcagttaattaaccagcctgcaatggaaattgtagtgcgctagaccacgcgctgctgatctgggccacgaactgcgcgcgtttgcatgcaggtggtctctgatcgcgaaccagctgccgggaaggacggacaacgacgtgaagaactactggaacacgaagctgagcaagaagctgcggcagcgcgggatcgaccccctcacccaccgccccatcgccgacctcatgcacagcatcggcgcgctggccatccgcccgccgcagccggcgacctcccctaacggctccgccgcctaccttcctgcgccagcgctcccgctcgtccacgacgtcgcgtaccacgccgccggaatgctgccgccgacgccggcgccgcagcggcaggtcgtcatcgcgcgcgtggaagcggacgcgcccgcgtcgccgacggagcacgggcacgagctcaagtggagcgacttcctcgccgacgacgccgccgccgcggcggcggccgcggccgaggcgcagcagcagctggccgttgttgggcagtaccaccacgaggccaacgccgggagcagcagcgctgcggccggcggtaacgacggttgtggcattgacgtcggcggcgacgacggcgcggcggcgttcatcgacgccatcctggactgcgacaaggagacgggggtggaccagctcatcgccgagctgctggccgacccggcctactacgcgggctcctcctcctcctcgtcgtccgggatgggcatgggctgggccggcatgggcctgctgaacgctgattaattaactcaagactgctttagtgtttgctatacctacttaccatcaattagtatgctggttga

SEQ ID NO: 5 - DNA, 1656 bp, Zea mays
gcgctcgccctaatgagctgttgatttgccggagggaagcagttgcgcgcgcgctctactatactgtcgccgccgccataaacaaagagggaaccagcgtctcttccctaatctaaccatctcctgcgtgattgacactaaccatgccgcggctagttaaatgacggggacggggtcacgccttcgtcgcgtgcctccacctccccccctcggcgcccccaacgacatgttgttaccgtggctgtggcagccggccggtctccttctccatccatatgtactggcagcatcgtatcacctttttctgcagcggtgatctcatctaggcgtcggtcagagctctctcgagctcgccagcggtggttggtcgtcgtcgtcgtcgtcgtcgatggggaggccgccgtgctgcgacaaggcgaacgtgaagaaggggccgtggacgccggaggaggacgccaagctgctggcctacacctccacccatggcaccggcaactggaccaacgtgccccagagagcaggtgatcgtgccgccgtgcaccatgcattttgttgttttgtttgtggctcatgacgaggcgctggcggacggcgcggcatgatgtgatatgatgcgcatggctcaagaggtgcggcaagagctgcaggctgaggtacaccaactacctgcgtcccaacctgaagcacgagaacttcacccaggaggaggaggacctcatcgtcaccctccacgccatgctcggaagcaggtacgcagtacagcgctgcagaatcatgttcatggccgtttgctttgatttgtttccgcggctgctactcctttccacacaacatgcatgcattcgcatcatccttcagcttcctcacctaggaaccggatagatccatcgtagtgctgcagataaatccgatttatcttctatctcaatgcggttttgaaactaagtatatcacattagatagtgttaattgctgaactgaagaagatctgaattatgaaagacacgtccgatcctgcagctcttacaaaggttttctctttttttaagaaaaaaaaatcggcctccatttaccgtaggtgattcttcctggacatttttgttccgcggcaaattaaatagtaattgaacctatgtttcacatgagaaaattgctagtaatcgggtgtttggaaatgattctgaatcttgcggacttaaatctgaaaccaatcgtcccaatgcaatttgctagagcaattgatctgttcatttccaatcaccaagccctagaaacggacgggacagctagttcagtagttcccgcatcagcgccattgctgatggatcgaacggctgacgcgaatgaaaacgacatgacaccgtcggggagatcgttggatgagttccgagcgataacgaactgtacgggcagtgacatacacaatgcgtgcgcgcatgcaaagttgattggaatccatgcgtccagctgatcactcgtagtcgtagtcatagttgctagggtaggtgatcttgagatgcatcttgatccctcgctagttagatcatgctatttgctgcagttaattaaccagcctgcaatggaaattgtagtgcgctagaccacgcgctgctgatctgggccacgaactgcgcgcgtttgcatgcaggtggtctc

SEQ ID NO: 6 - DNA, 963 bp, Zea mays
atggggaggccgccgtgctgcgacaaggcgaacgtgaagaaggggccgtggacgccggaggaggacgccaagctgctggcctacacctccacccatggcaccggcaactggaccaacgaacgtgccccaacgagcagggctcaagaggtgcggcaagagctgcaggctgaggtacaccaactacctgcgtcccaacctgaagcacgagaacttcacccaggaggaggaagacctcatcgtcaccctccacgccatgctcggaagcaggtggtctctgatcgcgaaccagctgccgggaaggacggacaacgacgtgaagaactactggaacacgaagctgagcaagaagctgcggcagcgcgggatcgaccccctcacccaccgccccatcgccgacctcatgcacagcatcggcgcgctggccatccgcccgccgcagccggcgacctcccctaacggctccgccgcctaccttcctgcgccggcgctcccgctcgtccacgacgtcgcgtaccacgccgccggaatgctgccgccgacgccggcgccgccccggcaggtcgtcatcgcgcgcgtggaagcggacgcgcccgcgtcgccgacggagcacgggcacgagctcaagtggagcgacttcctcgccgacgacgccgccgccgcggcggcggccgcggccgaggcgcagcagcagctggccgttgttgggcagtaccaccacgaggccaacgccgggagcagcagcgctgcggccggcggtaacgacggttgtggcattgccgtcggcggcgacgacggcgcagcggcgttcatcgacgccatcctggactgcgacaaggagacgggggtggaccagctcatcgccgagctgctggccgacccggcctactacgcgggctcctcctcctcctcctcctcctcgtccgggatgggctgggccggcatgggcctgctgaacgctgatta

SEQ ID NO: 7 - DNA, 943 bp, Zea mays
atggggaggccgccgtgctgcgacaaggcgaacgtgaagaaggggccgtggacgccggaggaggacgccaagctgctggcctacacctccacccatggcaccggcaactggaccaacgtgccccaacgagcagggctcaagaggtgcggcaagagctgcaggctgaggtacaccaactacctgcgtcccaacctgaagcacgagaacttcacccaggaggaggaagacctcatcgtcaccctccacgccatgctcggaagcaggtggtctctgatcgcgaaccagctaacgacgtgaagaactactggaacacgaagctgagcaagaagctgcggcagcgcgggatcgaccccctcacccaccgccccatcgccgacctcatgcacagcatcggcgcgctggccatccgcccgccgcagccggcgacctcccctaacggctccgccgcctaccttcctgcgccggcgctcccgctcgtccacgacgtcgcgtaccacgccgccggaatgctgccgccgacgccggcgccgccccggcaggtcgtcatcgcgcgcgtggaagcggacgcgcccgcgtcgccgacggagcacgggcacgagctcaagtggagcgacttcctcgccgacgacgccgccgccgcggcggcggccgcggccgaggcgcagcagcagctggccgttgttgggcagtaccaccacgaggccaacgccgggagcagcagcgctgcggccggcggtaacgacggttgtggcattgccgtcggcggcgacgacggcgcagcggcgttcatcgacgccatcctggactgcgacaaggagacgggggtggaccagctcatcgccgagctgctggccgacccggcctactacgcgggctcctcctcctcctcctcctcctcgtccgggatgggctgggccggcatgggcctgctgaacgctgatta

SEQ ID NO: 8 - DNA, 947 bp, Zea mays
atggggaggccgccgtgctgcgacaaggcgaacgtgaagaaggggccgtggacgccggaggaggacgccaagctgctggcctacacctccacccatggcaccggcaactggaccaacgaacgtgccccaacgagcagggctcaagaggtgcggcaagagctgcaggctgaggtacaccaactacctgcgtcccaacctgaagcacgagaacttcacccaggaggaggaagacctcatcgtcaccctccacgccatgctcggaagcaggtggtctctgatcgcgaaccagctaacgacgtgaagaactactggaacacgaagctgagcaagaagctgcggcagcgcgggatcgaccccctcacccaccgccccatcgccgacctcatgcacagcatcggcgcgctggccatccgcccgccgcagccggcgacctcccctaacggctccgccgcctaccttcctgcgccggcgctcccgctcgtccacgacgtcgcgtaccacgccgccggaatgctgccgccgacgccggcgccgccccggcaggtcgtcatcgcgcgcgtggaagcggacgcgcccgcgtcgccgacggagcacgggcacgagctcaagtggagcgacttcctcgccgacgacgccgccgccgcggcggcggccgcggccgaggcgcagcagcagctggccgttgttgggcagtaccaccacgaggccaacgccgggagcagcagcgctgcggccggcggtaacgacggttgtggcattgccgtcggcggcgacgacggcgcagcggcgttcatcgacgccatcctggactgcgacaaggagacgggggtggaccagctcatcgccgagctgctggccgacccggcctactacgcgggctcctcctcctcctcctcctcctcgtccgggatgggctgggccggcatgggcctgctgaacgctgatta

SEQ ID NO: 9 - protein, 100 aa, Zea mays
MGRPPCCDKANVKKGPWTPEEEDAKLLAYTSTHGTGNWTNVPQRAGLKRCGKSCRRLRYTNYPRPNLKHENFTQEEEDLIVTLHAMLGSRWSLIANQQTT

SEQ ID NO: 10 - protein, 104 aa, Zea mays
MGRPPCCDKANVKKGPWTPEEDAKLLAYTSTHGTGNWTNERAPESRAQEVRQELQAEVHQLPASQPEARELHPGGGGPHRHPPRHARKQVRSTALQNHVHGRLL

SEQ ID NO: 11 - protein, 100 aa, Zea mays; identical to SEQ ID NO: 9

SEQ ID NO: 12 - protein, 314 aa, Zea mays
MGRPPCCDKANVKKGPWTPEEDAKLLAYTSTHGTGNWTNVPQRAGLKRCGKSCRLRYTNYLRPNLKHENFTQEEEDLIVTLHAMLGSRWSLIANQLNDVKNYWNTKLSKKLRQRGIDPLTHRPIADLMHSIGALAIRPPQPATSPNGSAAYLPAPALPLVHDVAYHAAGMLPPTPAPPRQVVIARVEADAPASPTEHGHELKWSDFLADDAAAAAAAAAEAQQQLAVVGQYHHEANAGSSSAAAGGNDGCGIAVGGDDGAAAFIDAILDCDKETGVDQLIAELLADPAYYAGSSSSSSSSSGMGWAGMGLLNAD

SEQ ID NO: 13 - protein, 328 aa, Sorghum bicolor
MGRPPCCDKANVKKGPWTPEEDAKLLAYTSTHGTGNWTNVPQRAGLKRCGKSCRLRYTNYLRPNLKHENFTQEEEDLIVTLHAMLGSRWSLIANQLPGRTDNDVKNYWNTKLSKKLRQRGIDPITHRPIADLMHSIGALAIRPPQPASSSPNGGYLPAPALPLVHDVAYHAAGMLPPKTEQQQVVIARVDADAPASPTTTEHGQGQQLKWSDFLADDAAAAAAAAEAQQQQVVLGQYHHEASAVGAGSGVAVYGAGSSSSAAAAGGDVGGGGGGDDGAAAFIDAILDCDKETGVDQLIAELLADPAYYAGSSSSSSEMGWGMGLLNAD

SEQ ID NO: 14 - protein, 306 aa, Oryza sativa
MGRPPCCDKANVKKGPWTPEEDAKLLAYTSTHGTGNWTSVPQRAGLKRCGKSCRLRYTNYLRPNLKHENFTQEEEELIVTLHAMLGSRWSLIANQLPGRTDNDVKNYWNTKLSKKLRQRGIDPITHRPIADLMQSIGTLAIRPPPAAGAAPPPCLPVFHDAPYFAALQHQHQQQQVVTHVDADAPASPDSQHLQLNWSDFLADDAAGHGADAPAPQAALGQYQEGSAPAATAVVGGGRAFGDVDGASAGVGAGTDDGAGAASAFIDAILDCDKEMGVDQLIAEMLADPAYYGGGGGSSSSELGWGC

SEQ ID NO: 15 - DNA, 966 bp, Zea mays
tccatccggaaacgaaaccgctagagctaaggtctcaatcaacacagtgtcgagtgtggacaagttggtgctgctggactgctggcacatatacgaaaacgaaagcaggtcaggtggagagcagaggaggagccgtgccgcaggcagctattttatagctgctcgacgcgggtgctacacgcgtccagtgggaacggctgcctttctcggagcgcgtagcctggcgcgcgctggttggctccactctgcggcgttttttcaatttgtttttttactcctctttctgcggggaggcggcgagatgtcgggtggacggtggatcggcaaacgaggggcctgtcggtgtggacggtggagatgcactaagccttctttgagcaggtcacatcttttgcaagttcatggtgcccagtacgtagtggacagcagagaagctccgggcggggccaatgcaaaaccgatctggcggcgtcgatgtcgtgaaaaccgtcatgcccaactcgtgaaaattttaaaccccagcatcaccaggccgctagctccgtctctcaacgataagattacccacaccaacccgcgctcgccctaatgagctgttgatttgccggagggaagcagttgcgcgcgcgctctactatactgtcgccgccgccataaacaaagagggaaccagcgtctcttccctaatctaaccatctcctgcgtgattgacactaaccatgccgtggctagttaaatgacgggggcggggtcacgccttcgttgcgtgcctccacctccccccctcggcgcccccaacgacatgttgttaccgtggctgtggcagccggccggtctccttctccatccatatgtaccggcagcatcgtatcacctttttttctgcagcggtgatctcatctaggcgtcggtcagagctctctcgagctcgccagcggtggttggtcgtcgtcgtcgtcgtcgtcg